(19) United States
(12) Patent Application Publication
(10) Pub. No.: US 2012/0211373 A1
(43) Pub. Date: Aug. 23, 2012

(54) MICROFLUIDIC SYSTEM FOR MEASURING CELL BARRIER FUNCTION

(75) Inventors: Mohamed El-Sayed, Dexter, MI (US); Nicholas Douville, Saline, MI (US); Yi-Chung Tung, Keelung (TW); Shuichi Takayama, Ann Arbor, MI (US); Jack Dong Wang, Ann Arbor, MI (US)

(73) Assignee: THE REGENTS OF THE UNIVERSITY OF MICHIGAN, Ann Arbor, MI (US)

(21) Appl. No.: 13/401,461

(22) Filed: Feb. 21, 2012

Related U.S. Application Data
(60) Provisional application No. 61/445,232, filed on Feb. 22, 2011.

(51) Int. Cl. G01N 27/403; G01N 27/40; C12N 5/07

(52) U.S. Cl. 205/778; 435/401; 204/403.01; 204/403.06

(57) ABSTRACT

The present invention relates to devices, systems and methods for measuring cell barrier function. In particular, the present invention relates to microfluidic devices for use in in vitro models of the blood-brain barrier and modeling the transport across this barrier.

This application claims priority to provisional application 61/445,232, filed Feb. 22, 2011, which is herein incorporated by reference in its entirety.

### STATEMENT REGARDING FEDERALLY SPONSORED RESEARCH OR DEVELOPMENT

This invention was made with government support under HL084370 awarded by the National Institutes of Health. The government has certain rights in the invention.

### FIELD OF THE INVENTION

The present invention relates to devices, systems and methods for measuring cell barrier function. In particular, the present invention relates to microfluidic devices for use in in vitro models of the blood-brain barrier and modeling the transport across this barrier.

### BACKGROUND OF THE INVENTION

The endothelial cells lining the capillaries that supply the brain with oxygen and nutrients present a highly regulated transport barrier known as the blood-brain barrier (BBB). These endothelial cells are characterized by thick cell membranes, low number of endocytic vesicles, absence of fenestrae, and highly organized tight junctions that restrict molecular diffusion across the paracellular space (Rubin and Staddon, Annual Review of Neuroscience 22, 11-28 (1999); Persidsky et al., Journal of Neuroimmune Pharmacology 1, 223-236 (2006)). The integrity and function of the BBB is regulated by several environmental conditions including laminar flow, endothelial cell-to-cell contact (Deli et al., Cellular and Molecular Neurobiology 25, 59-127 (2004)), communication with other supporting cell types such as astrocytes, pericytes, microglia, and neurons, and the local concentration of secreted chemical factors (Abbott, Journal of Anatomy 200, 629-638 (2002)). Due to the presence of this highly functional barrier, only 2% of small-molecule drugs (<500 Daltons) can cross the BBB and achieve their effective therapeutic concentrations in the brain while nearly none of the existing large-molecule drugs can cross the BBB (Pardridge, Molecular Interventions 3, 90-105 (2003)).

Only a few central nervous system (CNS) disorders, such as depression, epilepsy, chronic pain, and affective disorders, respond to clinical treatments by the 2% of small-molecule drugs that are available; on the other hand, many more serious CNS disorders cannot be effectively treated by these small therapeutic molecules including Alzheimer disease, Huntington disease, stroke, brain cancer, brain and spinal cord injury, HIV infection of the brain, etc (Pardridge, Molecular Interventions 3, 90-105 (2003)). Because of limitations of the currently available drug molecules, the CNS pharmaceutics market needs to grow more than 500% to reach the cardiovascular drug market despite the fact that there are 4 times more people afflicted with CNS disorders than cardiovascular disorders (Pardridge, DDT 7, 5-7 (2002)).

The inability of conventional drug molecules to cross the BBB has, in some cases, forced drug development programs to go forward without the consideration of BBB transport at all. This led to the development of craniotomy-based drug delivery method where a hole is drilled in the head, and drugs are directly administered to the brain, thus bypassing the BBB altogether (Pardridge, Molecular Interventions 3, 90-105 (2003)). However, drug molecules administered this way tend to stay at the injection site and cannot effectively penetrate the brain parenchyma. With such limited penetration capability, only 1% of the brain volume is reached, making this method ineffective against most, if not all, brain diseases (Pardridge, Molecular Interventions 3, 90-105 (2003)). Further, craniotomy is an invasive method for drug administration that cannot be regularly used for treatment of chronic CNS disorders.

Clinical research has also shown that osmotic modification of the BBB can increase the delivery of drug molecules, specifically chemotherapeutic agents for the treatment of brain tumors. Typically, 1.4 M mannitol solution is introduced into the system by intracarotid infusion resulting in the opening of tight junctions at the BBB due to increased osmotic pressure at the apical side of the capillaries. This method, coupled with intravenous infusion of chemotherapeutic agents, has been found to prolong survival of patients with high-grade gliomas by 10-12 months (Gumerlock et al., Journal of Neuro-oncology 12, 33-46 (1992)); however, this therapeutic method is associated with cognitive deterioration or changes in the central nervous system (CNS) detectable by Magnetic Resonance Imaging (Roman-Goldstein, S. et al. American Journal of Neuroradiology 16, 543-553 (1995)).

In comparison to the limitations of methods such as the craniotomy-based drug delivery system and osmotic modification of the BBB, drug molecules that reach the systemic circulation after oral absorption or intravenous or intracarotid administration are more effective at penetrating the BBB if they can take advantage of the established transport mechanisms, which increases their therapeutic activity while minimizing side effects. Drug molecules capable of such maneuvers have the ability to reach nearly all of the neurons in the brain since every neuron is supported by its own capillary vessel (Pardridge, Molecular Interventions 3, 90-105 (2003)); however, it requires innovative drug-targeting systems that have the capability of traversing through the BBB. In order to develop such a system, an accurate and cost-effective in vitro BBB model that can be used repeatedly for drug screening and experimentation is needed.

### SUMMARY OF THE INVENTION

The present invention relates to devices, systems and methods for measuring cell barrier function. In particular, the present invention relates to microfluidic devices for use in in vitro models of the blood-brain barrier and modeling the transport across this barrier.

Embodiments of the present invention provide improved systems and methods that provide in vitro models of the blood brain barrier. The systems and methods described herein overcome many of the drawbacks of existing systems to provide cell monolayers that closely mimic in vivo endothelial monolayers such as those that comprise the blood brain barrier. The systems and methods described herein find use in research, clinical, and screening (e.g., drug screening) applications.

For example, in some embodiments, the present invention provides a system, comprising: a) a device comprising: i) a plurality of microfluidic channels; ii) a membrane (e.g., a semi-porous membrane) located internal to the microfluidic channels; and iii) a plurality of electrodes embedded in the microfluidic channels; and b) a plurality of cells adhered to the membrane or adhered to the base of microfluidic channels. The present invention is not limited to a particular cell type. In some embodiments, cells are endothelial cells such as brain endothelial cells (e.g., b.End3 cells or human brain vascular endothelial cells (HCMEC/D3)), astrocytes, glial cells, microglia, neurons or a mixture thereof. In some embodiments, the cells grow in a monolayer. In some embodiments, the system further comprises a circulatory component configured to circulate fluid through the microfluidic channels. In some embodiments, the circulatory component is a pump. In some embodiments, the pump further comprises valve that control the direction and/or speed of the flow of fluid. In some embodiments, the circulatory component further comprises a reservoir for storing fluids (e.g., buffers and other reagents for supporting cell growth). In some embodiments, the electrodes are Ag/AgCl electrodes. In some embodiments, the system further comprises a test compound (e.g., a drug that functions in the central nervous system or brain).

Further embodiments provide a method, comprising: a) contacting a device comprising a plurality of microfluidic channels, a membrane located internal to the microfluidic channels; and a plurality of electrodes embedded in the microfluidic channels with a plurality of cells, wherein the cells adhere to the membrane or the microfluidic channel; and b) culturing the cells, wherein the cells form a monolayer on the membrane. In some embodiments, a different cell type is co-cultured on the opposite side of the membrane or the bottom channel. In some embodiments, the method further comprises the step of measuring TEER across the membrane. In some embodiments, the method further comprises the step of contacting the membrane with a test compound (e.g., a drug that functions in the central nervous system or brain). In some embodiments, the method further comprises the step of measuring the transport of the test compound across the membrane.

Additional embodiments are described herein.

### DESCRIPTION OF THE FIGURES

FIG. 1 shows system design with an equivalent circuit model of embodiments of the present invention.

FIG. 2 shows a fabrication overview. (a) Device bonding. (b) Securing embedded electrodes. (c) Photograph of fabricated device.

FIG. 3 shows system characterization. (a) Impact of varying KCl concentration on impedance spectra. (b) Impact of varying electrolyte solution on impedance spectra.

FIG. 4 shows resolved TEER as a function of days since confluence for different cell lines (b.END.3 and MDCK-2). Both cell lines followed an increase-plateau decrease pattern similar to previously published models and significant differences between each of the three chosen cell lines could be resolved (a, b). Changes in TEER resulting from culturing a cell line not expected to form a tight junction were also measured. The electrical resistance across a C2C12 monolayer (c) displayed a minimal increase [<20 Ωcm2 in the positive direction]. The data from the individual chips are combined in (d).

FIG. 5 shows an illustration of endothelial cells constituting the BBB and their communication with pericytes, astrocytes, neurons, and microglial cells. a. Brain endothelial cells featuring glucose transporter 1 (GLUT-1), L-system for large neutral amino acids (LAT-1), and excitatory amino acid transporters 1-3 (EAAT1-3). b. Examples of bidirectional astrocytic interactions with endothelial cells.

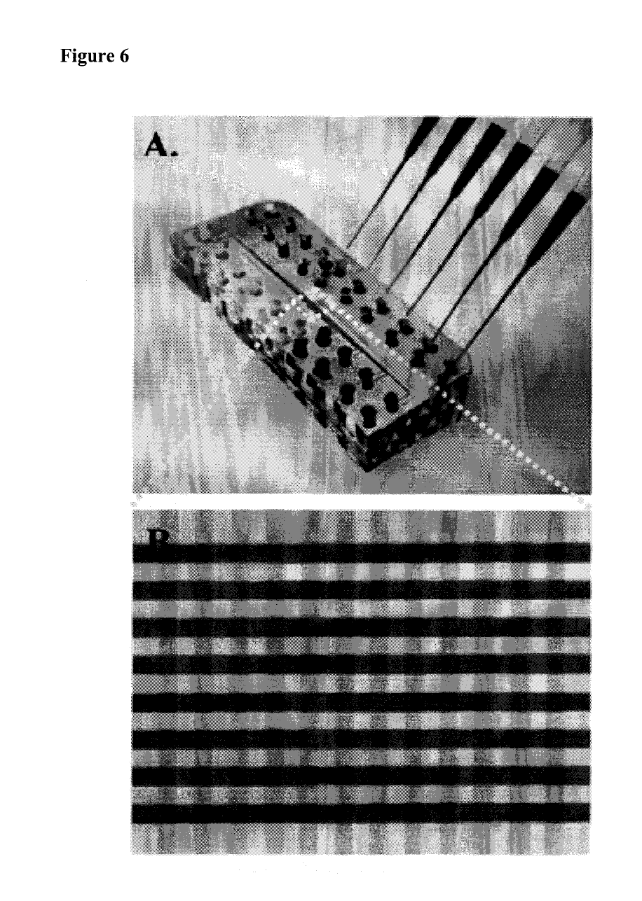

FIG. 6 shows a. An image of the device with a pipettor loading fluid into the reservoirs. b. A micrograph of the area where the layers intersect.

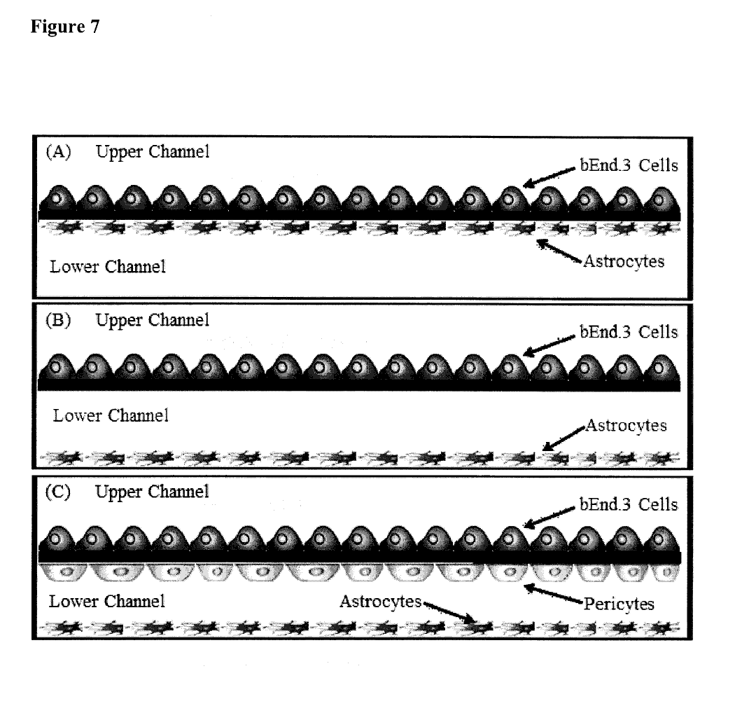

FIG. 7 shows schematic drawings of different co-culture systems; (A) shows b.End3 endothelial cells and astrocytes cultured on opposite sides of the porous membrane sandwiched between two microfluidic channels; (B) shows b.End3 endothelial cells cultured onto the porous membrane and the astrocytes cultured in the bottom of the lower channel; (C) shows b.End3 endothelial cells and pericytes cultured on opposite sides of the porous membrane sandwiched between two microfluidic channels and the astrocytes cultured in the bottom of the lower channel.

FIG. 8 shows the schematic drawing of layered PDMS channel configuration with fluorescent images of b.End3 cells cultured in these channels (40 mm×2 mm×0.2 mm) sandwiching polyester membrane filter (400 nm pore size) at 10× magnification stained with Live/Dead assay (Live/Dead Viability/Cytotoxicity Kit for mammalian cells, Invitrogen, Carlsbad, Calif.) at different culture time points.

FIG. 9 shows (A) Cell counts of confluent b.End3 monolayers at 10× magnification (1.17 mm×0.88 mm field view) as a function of culture time points (days). (B) Cell Shape Index (SI) measurements of confluent b.End3 monolayers at 4× magnification (2.87 mm×2 mm field view) as a function of culture time points (days). (C) Angle measurements along the direction of flow of confluent b.End3 monolayers at 4× magnification as a function of culture time points (days).

FIG. 10 shows (A) Microfluidic system used to measure transendothelial electrical resistance (TEER) with Ag/AgCl recording electrodes embedded on opposing sides of b.End3 cells cultured on a polyester porous membrane (400 nm pore size). (B) Comparison between TEER measurements of confluent b.End3 monolayers cultured inside 12-well transwells and layered microfluidic devices as a function of culture time points (days). (*: p<0.05; **: p<0.01)

FIG. 11 shows (A) Comparison between paracellular permeability of [14C]-mannitol through confluent b.End3 monolayers cultured inside 12-well transwells and layered microfluidic devices as a function of culture time points (days). (***: p<0.005) (B) Comparison between paracellular permeability of [14C]-inulin through confluent b.End3 monolayers cultured inside 12-well transwells and layered microfluidic devices as a function of culture time points (days). (C) Comparison between paracellular permeability of [14C]-dextran through confluent b.End3 monolayers cultured inside 12-well transwells and layered microfluidic devices as a function of culture time points (days). (D) Comparisons among [14C]-mannitol, [14C]-inulin, and [14C]-dextran permeability through confluent b.End3 monolayers cultured inside 12-well transwells as a function of culture time points (days). (E) Comparisons among [14C]-mannitol, [14C]-inulin, and [14C]-dextran permeability through confluent b.End3 monolayers cultured inside layered microfluidic devices as a function of culture time points (days).

FIG. 12 shows comparison between the efflux ratios (permeability_{BA}/permeability_{AB}) of confluent b.End3 monolayers cultured inside 12-well transwells and layered microfluidic channels as a function of culture time points (days) (A). FIG. 11B shows a comparison between P-glycoprotein transport of [3H]-dexamethasone through confluent b.End3 monolayers cultured inside 12-well transwells and layered microfluidic channels as a function of culture time points (days).

FIG. 13 shows the difference in mRNA levels of tight junction proteins (Occludin, Claudin-1, Claudin-3, Claudin-5) and cytoskeletal actin expressed by b.End3 cells cultured in conventional 12-well transwells and layered microfluidic devices after 7 days in culture.

FIG. 14 shows schematic drawing of automatic flow system for the dynamic microfluidic model of BBB.

### DEFINITIONS

To facilitate an understanding of the present invention, a number of terms and phrases are defined below:

The term “sample” in the present specification and claims is used in its broadest sense. On the one hand it is meant to include a specimen or culture. On the other hand, it is meant to include both biological and environmental samples. A sample may include a specimen of synthetic origin.

Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc.

Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

As used herein, the term “cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function. Test compounds comprise both known and potential therapeutic compounds. A test compound can be determined to be therapeutic by screening using the screening methods of the present invention. In some embodiments of the present invention, test compounds are agents (e.g. chemical or biological agents) that function in the CNS or agents that are useful in investigating the regulation or regulating the integrity and/or function of the blood brain barrier.

In some embodiments, test compounds are “drugs with unknown blood brain barrier passive capability.” As used herein, the term “drugs with unknown blood brain barrier passive capability” refers to drugs whose ability to cross the blood brain barrier is unknown. In some embodiments, “drugs with unknown blood brain barrier passive capability” are known to have therapeutic activity, while in other embodiments, the therapeutic activity of the drug is unknown.

### DETAILED DESCRIPTION OF THE INVENTION

The present invention relates to devices, systems and methods for measuring cell barrier function. In particular, the present invention relates to microfluidic devices for use in in vitro models of the blood-brain barrier and modeling the transport across this barrier.

Realizing the importance of in vitro BBB models, many groups have attempted to construct such models using both primary and immortalized cell lines. Primary endothelial cells isolated from bovine, human, porcine, and rodent are typically used in modeling the BBB (Deli et al., Cellular and Molecular Neurobiology 25, 59-127 (2004)). Among all such established models, one porcine model demonstrated relatively high trans-endothelial electrical resistance (TEER) and low permeability (Deli et al., supra), which are important characteristics of the BBB. Despite the ethical questions and the logistical difficulties of obtaining human brain tissue, there have been reports of established human BBB models; these models, however, are less robust than porcine models according to published data (Deli et al., supra). All primary models suffer from several disadvantages. Primary cells are expensive to obtain and the reconstitution process is time consuming. Furthermore, the homogeneity of obtained cells is difficult to determine, and can be easily contaminated with neighboring cell types such as the pericytes (Deli et al., supra).

In recent years, immortalized brain endothelial cell lines have been more commonly used than primary cell lines due to their ease of manipulation and reproducibility. There are currently more than 20 endothelial cell lines available and virtually all of them have been used to establish in vitro BBB models with published results (Deli et al., supra). Many of these models utilize a transwell system, which is the standard tool for in vitro drug screening. The major limitations of such models are: i) poor formation of tight junction proteins, which results in formation of leaky monolayers with low TEER and high paracellular permeability values; and ii) poor expression of functional transporters (e.g., the P-glycoprotein efflux pump) on the apical surface of cultured endothelial cells. Since these models did not generate BBB characteristics, it is very difficult to use them for BBB related studies. The present invention is not limited to a particular mechanism. Indeed, an understanding of the mechanism is not necessary to understand the present invention. Nonetheless, it is contemplated that the reasons for the failed mimicry of the restrictive transport properties of the BBB can be due to the formation of “loose” tight junctions between adjacent endothelial cells, lower expressions of specific carriers, and/or limited cell viability. This can be attributed to the lack of proper micro-environments and efficient integration of endothelial and neural supporting cells in a model that allow cell-to-cell communication necessary to induce the proper differentiation of brain capillary endothelial cells, formation of restrictive tight junctions, and functional expression of different transporters at the levels present in vivo.

In 2008, Cucullo and colleagues published a dynamic in vitro model of the BBB that utilizes astrocyte co-culture and pulsatile flow, both of which are important environmental factors that influence the development and regulation of BBB (Cucullo et al., Journal of Cerebral Blood Flow & Metabolism 28, 312-328 (2008)). The model demonstrated high TEER values similar to that of primary porcine endothelial cell culture model and low permeability measured by using paracellular transport markers such as mannitol and sucrose.

In the early 1980s, Stewart and colleagues established that the development of BBB is due to the response of endothelial cells to their micro-environments. They accomplished this by implementing chick capillaries in both quail brain graft and quail somite graft. They observed that chick caprillaries in quail brain graft displayed blood-brain barrier like properties such as the existence of numerous tight junctions and very limited number of pinocytotic vesicles; while chick capillaries in quail somite graft near the skeletal muscle contained large numbers of pinocytotic vesicles and few mitochondria (Cucullo et al., supra).

Embodiments of the present invention provide a physiologically relevant in vitro model of the BBB (e.g., by culturing brain microvascular endothelial cells within a microfluidic system that possess the capability of inducing physiological flow). By mimicking several biological micro-environments such as down-sizing the growth area, increasing surface-to-volume ratio, and utilizing flow-induced shear stress, a model of an in vivo micro-environments is provided under in vitro conditions that stimulates endothelial growth and differentiation with BBB-like properties. The devices and systems described herein provide several advantages over existing systems, for example: i) improved endothelial cell polarization and enhanced formation of tight junctions, ii) the ability to provide physiological flow parameters that influence tight junction and cytoskeletal protein expressions, and iii) minimizing the dilution of secreted chemical factors. These devices, systems and methods find use in cellular and molecular studies of the BBB (e.g., for research studies) and to evaluate different strategies to enhance the net transport of therapeutic agents into the brain (e.g., drug screening applications).

### I. Devices and System

As described above, in some embodiments, the present invention provides devices and systems for measuring barrier function. Exemplary device construction methods are described below.

### A. Devices

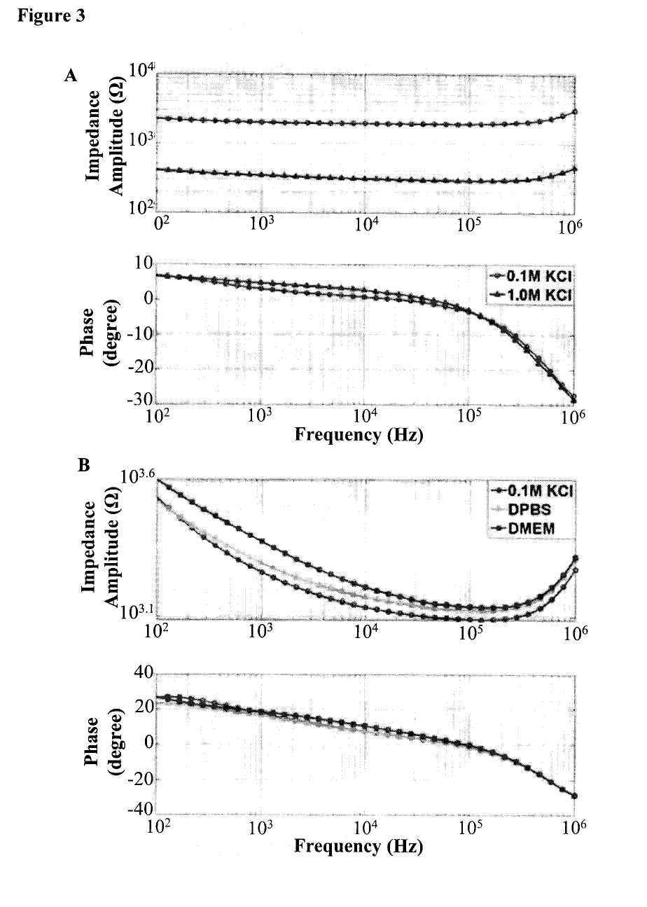

In some embodiments, devices comprise a plurality of microfluidic channels generated in a solid material. In some embodiments, devices comprise integrated electrodes (e.g., located on opposite sides of the device). In some embodiments, devices comprise a porous membrane (e.g., for cell culture). In some embodiments, devices comprise a top compartment comprising the top portion of the microfluidic channel and a bottom compartment comprising the bottom portion of the micrfluidic channel. In some embodiments, the top and bottom components are joined to form the complete device with microfluidic channels and electrodes above and below the membrane. An exemplary device is shown in FIG. 2.

In some embodiments, devices comprise a porous or semi-porous membrane. Any suitable membrane may be utilized. Examples include, but are not limited to, polyester or polycarbonate membranes.

In some embodiments, devices are constructed out of PDMS (e.g., using soft lithography). Soft lithography techniques as described for example by Duffy et al (Analytical Chem 70 4974-4984 1998; See also Anderson et al, Analytical Chem 72 158-64 2000 and Unger et al., Science 288 113-16 2000). Addition-curable RTV-2 silicone elastomers such as SYLGARD 184 Dow Corning Co. can be used for this purpose. In some embodiments, wells are generated in the cured polymer. In some embodiments, the wells are formed by punching a hole in a fabricated device and then forming a bottom deformable membrane. Manufacturing the substrate of elastomeric material facilitates a deformable well. The depth also depends, for example, on the extension possible for the actuators extendable protrusions.

The substrate may be of one layer or plurality of layers. The individual layers may be prepared by numerous techniques including laser ablation, plasma etching, wet chemical methods, injection molding, press molding, etc. Casting from curable silicone is most preferred, particularly when optical properties are important. Generation of the negative mold can be made by numerous methods all of which are well known to those skilled in the art. The silicone is then poured onto the mold degassed if necessary or desired and allowed to cure. Adherence of multiple layers to each other may be accomplished by conventional techniques.

An exemplary method of manufacture of some devices employs preparing a master through use of negative photoresist SU-8 50 photoresist from Micro Chem Corp Newton Mass. The photoresist may be applied to glass substrate and exposed from the uncoated side through suitable mask. Since the depth of cure is dependant on factors such as length of exposure and intensity of the light source features ranging from very thin up to the depth of the photoresist may be created. The unexposed resist is removed leaving a raised pattern on the glass substrate. The curable elastomer is cast onto this master and then removed. The material properties of SU-8 photoresist and the diffuse light from an inexpensive light source can be employed to generate devices. Short exposures tend to produce radiused top while longer exposures tend to produce flat top with rounded corners. Longer exposures also tend to produce wider channels.

In a typical procedure, a photoresist layer is exposed from the backside of the substrate through mask, for example, photoplotted film, by diffused light generated with an ultraviolet UV transilluminator. In some embodiments, wells are generated due to the way in which the spherical wavefront, created by diffused light penetrates into the negative photoresist. The exposure dose dependent change in the SU-8 absorption coefficient is 3985m-1 unexposed to 9700 m-1 exposed at 365 nm limits exposure depth at the edges. The exact cross-sectional shapes and widths of the fabricated structures are determined by a combination of photomask feature size exposure 20 time/intensity resist thickness and distance between the photomask and photoresist.

Although backside exposure makes features which are wider than the size defined by the photomask, and in some cases smaller in height compared to the thickness of the original photoresist coating, the change in dimensions of the transferred patterns is readily predicted from mask dimensions and exposure time. The relationship between the width of the photomask patterns and the photoresist patterns obtained is essentially the linear slope of beyond certain photomask aperture size. This linear relationship allows straightforward compensation of the aperture size on the photomask through simple subtraction of constant value. When exposure time is held constant there is a threshold aperture size below which incomplete exposure will cause the microchannel height to be lower than the original photoresist thickness. Lower exposure doses will make channels with smoother and more rounded cross-sectional profiles. Light exposure doses that are too slow or photoresist thicknesses that are too large however, are insufficient in penetrating through the photoresist, resulting in cross-sections that are thinner than the thickness of the original photoresist.

In some embodiments, multiple components of two component devices (e.g., a top portion and a bottom portion) are joined used PDMS polymer, although other methods are contemplated.

### B. Systems

Embodiments of the present invention provide microfluidic systems and kits comprising devices described herein, alone or in combination with cells (e.g., endothelial cells), reagents for culturing cells, test compounds (e.g., drugs), etc.

In some embodiments, the present invention provides systems that model the blood brain barrier. For example, in some embodiments, systems comprise devices (e.g., those described above) with membranes comprising endothelial cells such as, for example, primary or immortalized brain capillary endothelial cells or brain endothelial cell lines. Exemplary cell lines include, but are not limited to, b.End3 cells, human brain vascular endothelial cell line (HCMEC/D3), astrocytes, microglia, pericytes, neurons or a combination thereof. In some embodiments, multiple cell types are co-cultured or different cell types are cultured on different portions of the membranes or microfluidic channels.

In some embodiments, cells are seeded (e.g., at a density of 6,000-25,000 (e.g., 8000) cells/channel, although other densities are contemplated. In some embodiments, growth medium is changed on a regular basis (e.g., every 12 hours, although other intervals are contemplated) to promote long term cell culture.

In some embodiments, brain endothelial cells growing on a membrane or in microfluidic channels of devices of embodiments of the present invention exhibit properties of in vivo blood brain barriers. For example, in some embodiments, cell monolayers exhibit tight junctions, high TEER values and permeability similar to that of in vivo cell monolayers.

In some embodiments, system comprise embedded electrodes (e.g., for TEER measurements). In some embodiments, electrodes are Ag/AgCl electrodes, although other electrodes are contemplated.

In some embodiments, systems are configured for static culture systems. In such embodiments, cell culture medium is changed at time intervals (e.g., those described herein).

In some embodiments, systems comprise components for dynamic flow cell culture. Dynamic cell culture provides flow induced shear stress (e.g., comparable to that experienced by endothelial cells in vivo). In some embodiments, systems have a circulatory component that circulates buffers or other fluids. In some embodiments, the circulatory component is a pump. In some embodiments, the pump comprises a valve that controls the direction and/or speed of the flow and a reservoir for storing fluids to be circulated.

In some embodiments, system and/or kits further provide reagents necessary, sufficient or useful for using the systems and devices for research and clinical uses. Examples of reagents include, but are not limited to, cell culture buffers, test compounds, controls, detection reagents, etc.

In some embodiments, systems are configured for use in high throughput (e.g., highly parallel) methods. For example, in some embodiments, systems include robots, automated sample and reagent handling components and the like.

In some embodiments, systems (e.g., high throughput systems) include software components. In some embodiments, software is configured to, for example, manage reagent and test compounds delivery and removal, including dynamic flow reagents, collect data, perform quality control and quality assurance and analyze data.

### II. Uses

The devices and system of embodiments of the present invention find use in a variety of applications, including, but not limited to, those disclosed herein.

In some embodiments, the devices, systems, and methods of embodiments of the present invention find use in research applications. For example, in some embodiments, devices and systems find use in drug screening application. The devices and systems described herein find particular use in the screening of drugs that function in the brain and thus optimally cross the blood brain barrier (e.g., drugs that act on the central nervous system). In some embodiments, brain endothelial cells (e.g., those disclosed herein) are placed in a device of embodiments of the present invention. Cells are then incubated with a variety of test compounds. Test compounds are assayed for their ability to cross the endothelial cell monolayer.

Test compounds (e.g., drugs) are assayed for their ability to cross the endothelial cell monolayer using a variety of methods. For example, in some embodiments, TEER values are used to measure integrity of membranes. In some embodiments, test compounds are labeled (e.g., with a radioactive isotope or other labeling methods) and assayed for their ability to cross the cell monolayers (e.g., using known methods).

In some embodiments, test compounds are further screened for their ability to exert a biological effect on the cells contained in the device.

In some embodiments, high throughput drug screening methods are utilized. For example, in some embodiments, arrays of devices as described above are utilized in the high throughput screening of large numbers of candidate therapeutics (e.g., drugs that act by crossing the blood brain barrier).

### EXPERIMENTAL

The following examples are provided in order to demonstrate and further illustrate certain preferred embodiments and aspects of the present invention and are not to be construed as limiting the scope thereof.

### Example 1

### A. Materials and Methods

Materials and Reagents. Poly(dimethysiloxane) (PDMS) (Sylgard 184) was purchased from Dow Corning (Midland, Mich.). SU-8 2150 for two-step positive photoresist features was purchased from MicroChem. Co. (Newton, Mass.). Polytester membranes were purchased from Corning Inc. (Corning, N.Y.). All cell lines were purchased from ATCC (Manassas, Va.). Ag/AgCl electrodes of 500 μm diameter were purchased from World Precision Instruments (Sarasota, Fla.).

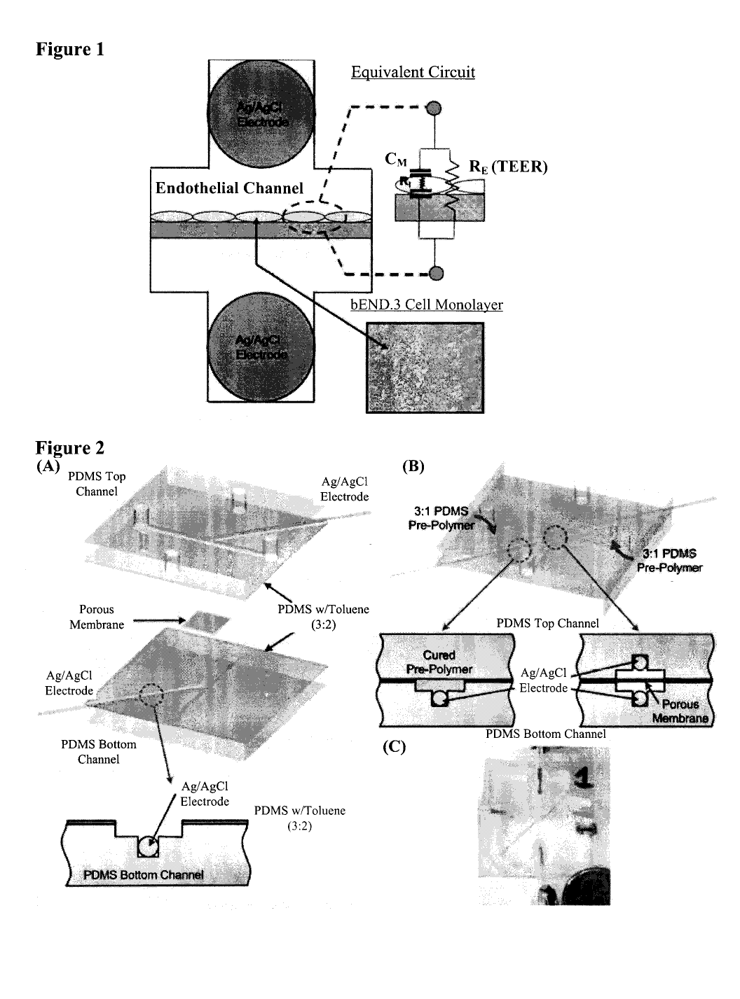

Device Design and Fabrication. The device is composed of two layers of PDMS microfluidic channels, which are designed with integrated microgrooves that permit the physical registration of two electrodes on opposite sides but in immediate proximity to a porous membrane on which cells are cultured for impedance measurement (Tung et al., J. Sens. Actuators, B 2004, 98, 356-367). The porous membrane on which cells attach and grow (initial diameter 24 mm and with a thickness of 10 μm) was first cut from the TransWell casing using a scalpel and trimmed to a size slightly larger than the area of overlap between the upper and lower channels (2 mm×2 mm). The cut membrane was then sandwiched between two PDMS channels. The elastomeric nature of the PDMS material combined with sealing of any crevices by PDMS mortar (Chueh et al., Anal. Chem. 2007, 79, 3504-3508), allows sealing of the channel despite the 10 μm thickness of the membrane. FIG. 1 provides an overview of the device design. The deposition-free integration of metallic electrodes alleviates any delaminating problem between metallic thin films and PDMS surfaces. Moreover, the fabrication process can be performed in the ambient environment without using sophisticated thin film deposition instruments in environment-controlled cleanrooms. Such microfluidic device design is capable of providing more functionalities and robust measurement results without increasing fabrication complexity.

The microfluidic channels on each PDMS layer were designed to have cross-sectional areas of 2000 μm (width) by 200 μm (height) and an additional channel groove (500 μm×500 μm) intersecting the main channel at a 45° angle for the measurement electrodes as shown in FIG. 2. The fabrication of the PDMS chips was conducted using a soft lithography technique. A silicon wafer with two-step positive relief features fabricated using negative tone photoresist (SU-8 2150, MicroChem. Co., Newton, Mass.) patterned by two separate photolithography steps was used as a mold. The mold was then silanized with tridecafluoro-1,1,2,2-tetrahydrooctyl-1-trichloro-silane (United Chemical Technologies, Bristol, Pa.) in a desiccator for more than 30 min at room temperature to prevent unwanted bonding. PDMS (Sylgard 184, Dow Corning) prepolymer mixed at a weight ratio of 12 (prepolymer)/1 (curing agent) was poured on the mold and cured overnight. The cured PDMS layers were removed from the wafer, and reservoir holes were punched within the upper PDMS layer allowing access to both the upper and lower channels (FIG. 2). Ag/AgCl recording electrodes with a diameter of 500 μm (World Precision Instruments, Sarasota, Fla.) were fit into the 500 μm×500 μm side channel and held by elastomeric tension through the sealing process. The electrodes were loaded through the entire length of the side channel but did not completely enter the main channel so that the confluency of the cell monolayer could be easily imaged. A 24 mm diameter (10 μm thickness) semiporous polyester membrane with pore size of 400 nm (Corning Inc., Corning, N.Y.) was cut into square sheets slightly greater than the overlapping area between the upper and lower channels (2 mm×2 mm) to completely cover the overlapping region. A PDMS mortar solution (3 toluene/2 PDMS by weight) was spun onto a glass microscope slide at 500 rpm for 5 s followed by 1500 rpm for 60 s to spread the mortar evenly along the entire surface of the glass slide. The upper and lower slabs of PDMS were contact printed onto the glass slide contact spreading the PDMS/toluene mortar onto the flat substrate while sparing the recessed channel features. The edges of the polyester membrane were also “stamped” onto the PDMS/toluene mortar before being placed over the overlapping region between the upper and lower slabs of PDMS to seal the channels (Chueh et al., supra). After sealing the layered channels, PDMS prepolymer at a weight of 3 (prepolymer)/1 (curing) agent was injected into the side channel using an 18 gauge blunt syringe needle. PDMS flow from the syringe was limited only to the side channel and flow was stopped before the PDMS prepolymer could flow into the main “cell culture” channels or immerse the tip of the electrodes. Flow was discontinued by holding pressure constant within the loading syringe containing PDMS prepolymer (sealed via elastomeric tension with the inlet reservoir punched into the PDMS). To further restrict PDMS flow into the main channels, the chip was elevated so that the main channel (and electrode tip) remained at a higher point than the side channel. The PDMS was allowed to cure for 24 h at room temperature, and the process was repeated for the second electrode.

Cell Seeding and Culture. Epithelial (MDCK-2) and endothelial (b.END.3) cell lines were selected because they offered well characterized and accepted TEER values to compare with the experimental results. Since mouse myoblast cells (C2C12) are not expected to form tight junctions, they were selected as a control cell line to demonstrate measurement variability inherent to the system or any impedance drift resulting from prolonged cell culture. All cell lines were cultured in endothelial growth media (DMEM with 10% fetal bovine serum and 1% penicillin/streptomycin) prior to seeding in the microfluidic chip. The microfluidic chip was exposed to plasma oxygen for 5 min to make hydrophilic. Prior to cell seeding, fibronectin solution (Invitrogen) at a concentration of 100 μg/mL in phosphate buffered saline (PBS) was injected into the upper (endothelial) channel to promote cell adhesion. The lower channel was preloaded with growth media. Since the fibronectin solution adheres to the porous membrane as well as the surrounding PDMS microchannel walls, cells can adhere and divide along the channel walls; however, due to the orientation and geometry of the system and gravity-driven pumping mechanism, the vast majority of cells adhere directly to the porous membrane. The fibronectin solution remained in the upper channel for 30 min at 25° C. under ultraviolet light to sterilize the system. After UV sterilization, sterile techniques and conditions were maintained. Following 30 min of fibronectin coating, the channel was completely washed with growth media to remove the nonadsorbed fibronectin and filled with growth media until seeding. Cells were trypsinized, centrifuged, and resuspended in 1 mL of growth media (to a concentration ˜10⁵ cells/mL). The cell suspension was then flowed through the upper channel until the pressure gradient between the inlet and outlet equilibrated, eliminating flow. The cells were allowed to adhere under this static condition (no flow) for ˜12 h before fresh media was flowed through the channel. The growth media was completely replaced with fresh media approximately every 12 h for the duration of the experiment. Immediately after seeding and for the duration of the culture time, the entire microfluidic system was kept in an incubation chamber (37° C., 5% CO₂). After successful cell seeding, cells rapidly divided until confluence. After reaching confluence, cells were successfully maintained within the microfluidic system for over 7 days of experimental measurement.

Impedance Spectroscopy. Impedance spectra were taken using an Autolab potentiostat/galvanostat (EcoChemie). Alternating current of amplitude 0.1 V was passed between the two embedded Ag/AgCl electrodes in the frequency range from 10 Hz to 1.00 MHz, yielding a total of 64 impedance measurements (8 per decade spaced logarithmically). The total time for a single frequency scan was approximately 7 min.

Resolving TEER from Circuit Model. In order to resolve the TEER value from the experimental impedance spectra, the control impedance spectra (taken from the same chip just before cell seeding) was subtracted from the measured impedance spectra (with cells) to eliminate the effect from electrolyte, membrane, and electrode-electrolyte interfaces and simplify the analysis. An equivalent lumped element circuit model (as shown in FIG. 1) was used to simulate the cell monolayer electrical behavior. The circuit is constructed by three components, where RE, RI, and CM represent extracellular (TEER) and intracellular resistances and a membrane capacitance, respectively (Kunii et al., Proc. 20th Annu. Int. Conf IEEE EMBS 1998, 20, 3108-3111; Kim et al., J. Appl. Physiol. 2004, 10, 1152-1180; Lewis et al., J. Membr. Biol. 1982, 67, 45-53). In order to resolve the value of each component, a MATLAB (The MathWorks, Inc., Natick, Mass.) code using its optimization tool box to estimate the values was developed. In the MATLAB code, the objective minimized the absolute difference between the simulated and experimental impedance values under various frequencies. The TEER value determined by the MATLAB algorithm was then normalized for surface area of the cell monolayer (specifically 0.04 cm²) by multiplying the total impedance yielding reported values in units of Ωcm².

### B. Results

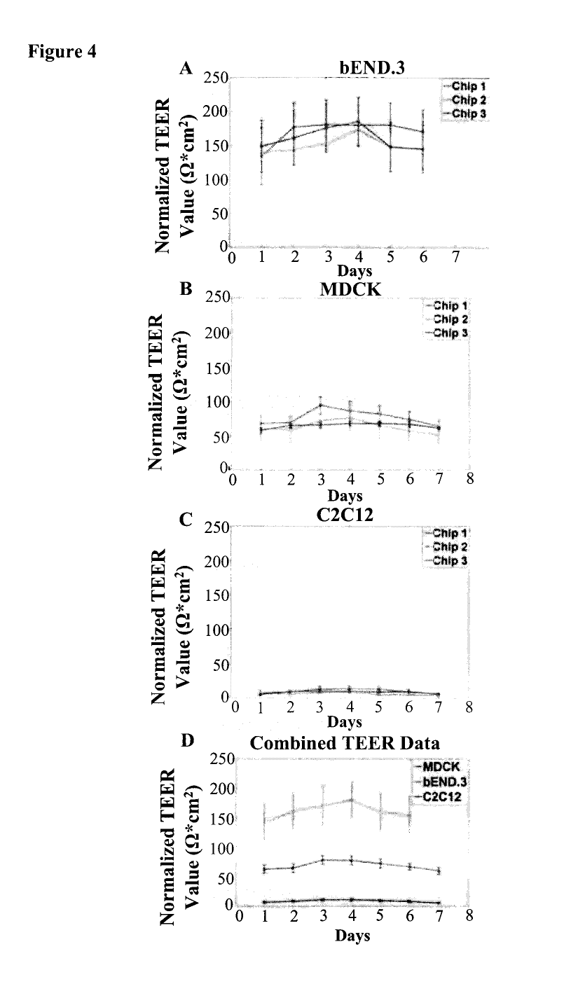

Characterization of System. Prior to seeding cells, the baseline impedance of the system (electrodes, electrolyte, and polyester membrane) was characterized. In initial experiments, the electrolyte solution contained within the microchannel as well as switched from a low-impedance polyester membrane to a high impedance PDMS membrane was varied. Increasing the concentration of the KCl electrolyte from 0.1 to 1.0 M resulted in a frequency-independent impedance decrease of greater than 1.5 kΩ (FIG. 3a). Replacing the porous polyester membrane with a PDMS membrane of similar thickness but without pores (˜10 μm) increased the impedance of the system from 100 kΩ (polyester) to the MΩ (PDMS) range. The PDMS membrane also contributed a capacitive effect to the impedance spectra. These experiments verified that the system correctly detected expected impedance changes. The impact of fibronectin (FN) treatment on the baseline impedance of the system was also tested. Baseline system impedance was recorded for three chips filled with PBS. Next, fibronectin in PBS (at a concentration of 100 μg/mL) was loaded into the system, allowing the fibronectin to deposit for 30 min, before the channel was flushed with fresh PBS. These results showed that FN treatment had a minimal impact on the impedance of the system. The impedance of a channel filled with endothelial growth media (DMEM), phosphate buffered saline (PBS), and 0.1 M KCl (no fibronectin was added to the system during these comparison controls) was also measured. The impedance of the system containing 0.1 M KCl had the lowest impedance; the PBS had intermediate impedance, and the growth media had the highest impedance (FIG. 3b). This experiment demonstrated the recording through channels containing endothelial growth media, eliminating the need for a specifically conductive electrolyte solution for cell recordings.

Traditional recording systems record impedance values at either DC current or a limited number of AC frequencies (Cereijido et al., J. Cell Biol. 1978, 77, 853-880; Giaever et al., Proc. Nat. Acad. Sci. U.S.A. 1984, 81, 3761-3764; Stamatovic et al., J. Biol. Chem. 2009, 284, 19053-19066). Measuring at high frequency minimizes but does not completely eliminate impedance resulting from the capacitive cell membrane. While sufficient for taking TEER measurements across a large area such as in TransWell cultures, particularly when the TEER values are large, measuring impedance across the full spectrum of AC frequencies enables the characteristic of the cell monolayer to be more accurately modeled and resolved (Kottra et al., Pflugers Arch. 1984, 402, 409-420; Erben et al., J. Biochem. Biophys. Methods 1995, 30, 227-238; Wegener et al., J. Biochem. Biophys. Methods 1996, 32, 151-170). The smaller microfluidic system is inherently prone to lower signal-to-noise ratios. For example, the surface area of the system is 0.04 cm² compared to a surface area of 4.91 cm² in the 6-well TransWell culture systems most commonly used for TEER measurements (and 0.143 cm² even in 96-well TransWell cultures which are considered very small). Thus, in addition to placing and immobilizing electrodes close to the cells, impedance was measured at over 64 frequency points and used to find the best fit to the entire equivalent circuit (FIG. 1) and resolve out factors that can vary independently of TEER.

A relatively large variability in impedance between different chips was found, most likely due to variance in the distance between the two electrodes caused by alignment variations of the upper and lower channel. It is estimated that for every millimeter difference in the distance between the two recording electrodes, the resistance changes by approximately 2.5 kΩ (Lee et al., Curr. Appl. Phys. 2008, 8, 696-699). Variability between different measurements within a single chip (where the electrodes remain fixed) was minimal. Thus, in the experimental TEER measurements, the baseline impedance of each individual chip was characterized prior to endothelial cell seeding and subtracted from the impedance of the cell monolayer within the chip. This allowed a circuit model to be fit that accounted for contribution of the cell monolayer.

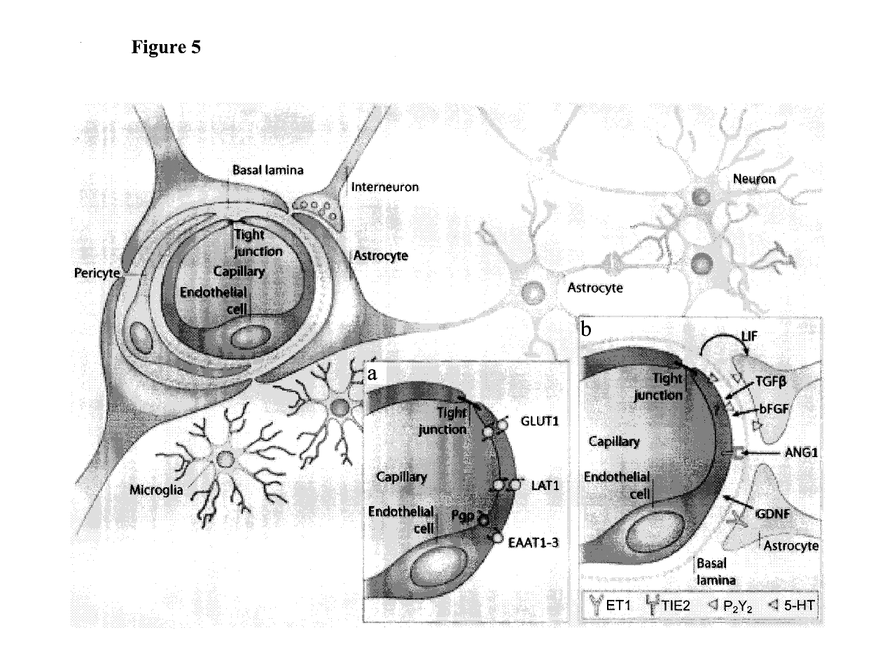

Establishing a Baseline Measure. As an experimental control and to establish a baseline measure, the impedance of the system was measured for 1 week under two conditions: (i) without cells (acellular) and (ii) with cells not expected to form tight junctions (C2C12 cell culture). In both conditions, the media was replaced with fresh media twice per day. Although no cells were cultured in the first control, TEER values were fit using the MATLAB algorithm and the experimental data was normalized to total membrane surface area. Measurements of the system containing growth media without cells showed relatively minimal change in TEER (<10 Ωcm² in either the positive or negative direction) when compared to the magnitude of TEER changes which result from tight junction formation in endothelial and epithelial cell lines (˜100 Ωcm² in the positive direction). Furthermore, the absence of either a positive trending TEER (from protein deposition on the electrodes) or negative trending TEER (if the Ag/AgCl electrodes were being reconditioned during testing) through 7 days of measurements (the first day is the baseline) indicate that the system is stable for long-term experimental measurements in serum-containing media. The present invention is not limited to a particular mechanism. Indeed, an understanding of the mechanism is not necessary to practice the present invention. Nonetheless, it is contemplated that because the TEER fluctuations do not trend in either the positive or negative direction, these changes indicate the baseline level of noise or error in the system resulting from recording equipment as well as other variables such as temperature fluctuation in the incubator, precise attachment of system to recording equipment, or resistivity of the DMEM solution. Next, changes in TEER resulting from culturing a cell line not expected to form tight junction were measured. The electrical resistance across a C2C12 monolayer (FIG. 4c) displayed a minimal increase (<20 Ωcm² in the positive direction) indicating that the physical presence of cells or cellular byproduct results in a nontrivial increase in monolayer resistance despite the theoretical absence of tight junctions which is able to be resolved from TEER changes resulting from tight junction formation.

TEER as a Function of Time with Epithelial and Endothelial Cells. MDCK-2 and b.END.3 cells were seeded onto the fibronectin-treated membrane. Impedance spectra were taken each day for seven consecutive days. Because temperature influences the impedance of the growth media, measurements were consistently taken 30 min after fresh media was loaded into the system and the system was kept incubated at 37° C. before and between all measurements. TEER values were resolved from the impedance spectra taken on each day following seeding using a best-fit circuit algorithm. Data showed that TEER values increased with each day of growth from day 1 to day 3 to 4 before reaching a plateau and even decreasing slightly. The error bars depicted in FIGS. 4a,b,c are the standard deviation of the 9 TEER values that can be resolved on each day, for each cell type. The experimental data agreed with the accepted TEER range of 150-200 Ωcm² for b.END.3 cells (Koto et al., Am. J. Path. 2007, 170, 1389-1397) and 50-150 Ωcm² for MDCK-2 cells (Richardson et al., Biochim. Biophys. Acta 1981, 673, 26-36). Additionally, both cell lines followed an increase-plateau-decrease pattern similar to previously published models, and significant differences between each of the three chosen cell lines could be resolved (FIG. 4).

Dynamic Impedance Changes in Response to Chemical/Protein Treatment. To demonstrate that the system can detect dynamic impedance changes in response to chemical factors, the impedance of a b.END.3 cell monolayer was measured; then, the cell monolayer was treated for 30 min with TritonX-100 (0.1% in DMEM) (van de Ven et al., J. Biomed. Opt. 2009, 14 (2), 011012-1-10), before the system was flushed with fresh DMEM and the impedance was re-measured. The experimental results showed that the impedance of the entire system decreased significantly at all frequency points following treatment with TritonX-100. The change in TEER (paracellular resistance) was also resolved using the MATLAB circuit fitting algorithm and found to decrease from 161.51±0.755 Ωcm² to 39.73±1.925 Ωcm².

Furthermore, a change in the impedance at all frequency points following trypsinization was demonstrated. The decrease in impedance amplitude shows that the system and technique are capable of detecting changes in response to chemical or protein treatment in real time.

Comparison to Existing TEER Measurement Systems. To enable reliable TEER measurement across the small cell culture area within microfluidic channels, the system incorporates features adapted from multiple advances that have been made over the years for macroscopic TEER measurement systems (Deli et al., Cell. Mol. Neurobiol. 2005, 25 (1), 59-127). The most widely used system for measuring TEER in vitro is the commercially available TransWell culture environment and the “chopstick-style” recording electrode (Mishler et al., Am. J. Physiol. Renal Physiol. 1990, 258 (6), F1561-F1568; Hurst et al., J. Cell. Physiol. 1996, 167, 81-88). For more reproducible and stable TEER measures of leaky cell barriers with lower TEER values, improved recording chambers with embedded electrodes have been constructed. An example is the Endohm chamber, where electrodes are placed immediately above and beneath the membrane to minimize resistance from the media and allow a more uniform current density to flow across the membrane. The microfluidic chamber incorporates a similar concept by embedding electrodes immediately above and below the cell culture membrane inside the channels. Another enhancement in measurement is represented by the cellZscope, which can measure the impedance across a wide spectrum of frequencies, rather than the typical one frequency, to give more detailed measures of barrier properties. The microfluidic TEER measurement system also acquires impedance spectra at 64 frequencies to obtain more accurate TEER values (Seebach et al., J. Lab. Invest. 2000, 80 (12), 1819-1831). Giaever and colleagues developed an alternative system that does not use porous membranes to measure endothelial barrier function of cells directly grown on gold electrodes (Giaever et al., Proc. Nat. Acad. Sci. U.S.A. 1984, 81, 3761-3764). This system has the advantage of enabling measurements of cell barrier functions over very small areas. Initial attempts to mimic this system by forming gold electrodes inside PDMS microchannels revealed that the flexibility of PDMS led to cracking of gold and limited the conductivity (Tiruppathi et al., Proc. Nat. Acad. Sci. U.S.A. 1992, 89, 7919-7923). The membrane embedded microfluidic TEER system described herein enables measurements to be made over relatively small cell culture areas. Additionally, by keeping the membrane sandwiched multilayer channel structure, the system enables basal cell treatment that is critical for optimal signaling in some cellular systems (Song et al., PLoS ONE 2009, 4 (6), e5756-e5756). The reported TEER values from another device (Harris et al., Proc. IEEE 2nd Annu. Northeast Bioeng. Conf. 2002,28, 1-2) were very low due to cell culture. Hediger and colleagues have also developed microfabricated systems for characterizing epithelial cell tissues (Hediger et al., Sens. Actuators, B 2000, 63, 63-73; Hediger et al., Biosens. Bioelectron. 2001, 16, 689-694). In this system, a nanoporous polycarbonate membrane is glued between two microscale reservoirs. This design is limited by multiple fabrication steps and a multilayered modular arrangement.

Comparison to Existing Techniques for Taking Electrical Recordings with Microfluidic Systems. There have been several reported methods for integrating electrodes within microfluidic systems (Siegel et al., Adv. Mater. 2007, 19, 727-733; Gray et al., Adv. Mater. 2004, 16, 393-401; Lacour et al., IEEE Electron Device Lett. 2004, 25, 792-794). In a method developed by Garcia and Henry, a gold wire was aligned within a perpendicular microfluidic channel and sealed using superglue and conducting paint (Garcia et al., Anal. Chem. 2003, 75, 4778-4783; Garcia et al., Analyst 2004, 129, 579-584). Electroplating and electroless deposition have been used to fabricate recording electrodes within microfluidic systems but are limited by multiple slow and costly lithographic steps and the requirement for a smooth rigid substrate (Polk et al., Sens. Actuators, B 2006, 114, 239-247). Die, impression, and injection molding have also proven useful for very specialized approaches but are limited by the high cost and complexity of the system (Martin et al., Anal. Chem. 2000, 72, 3196-3202). The systems described herein can be easily fabricated using standard soft-lithography techniques and utilize readily available and standardized electrodes which enable this technique to be easily expanded to other models and systems.

### Example 2

### Fabrication of Layered Microfluidic Channels and the Establishment of Cell Culture Protocol

Layered microfluidic channels were fabricated using soft lithography (Duffy et al., Analytical Chemistry 70, 4974-4984 (1998)). Briefly, PDMS prepolymer was mixed with curing agent at a weight ratio of 10 (prepolymer):1 (curing agent) and was cast onto two 4 inch silicon wafer containing a positive relief pattern that was 200 μm thick, one for the top layer and one for the bottom layer. The mixture was cured at 60° C. for 2 hours, then the cured PDMS layer was peeled from the silicon wafer. Access holes were punched with a 16 gauge blunt syringe (1.65 mm outer diameter) forming the inlet and outlet holes for each channel. In order to glue the top and the bottom layers together, PDMS mortar layers were created using PDMS and toluene mixed at 3 (PDMS):2 (toluene) weight ratio. The mixture was well mixed using a vortex mixer and it was allowed to sit for 5 minutes to remove bubbles. The toluene diluted PDMS prepolymer was then spin-coated on a clean glass cover slide for 1 minute to generate a thin mortar layer. The PDMS substrates were placed onto the glass slide spin-coated with adhesive PDMS mortar and allowed to stay in contact for 30 seconds. Polyester membranes with 200 nm pore size were used to cover the overlapping region of the top and bottom layers and both layers were aligned and glued together. The combined pieces were cured again for 1 hour until the PDMS mortar was completely hardened. 100 μl pipette tips were inserted into the inlets and outlets, acting as medium reservoir for the channel; they were then glued to the channel with PDMS. Before use, the layered PDMS channels were exposed to plasma oxygen for 5 minutes and fibronectin solution was flowed through the channels at 25 μg/ml. The solution was allowed to stay inside the channels under UV for 24 hours for coating and sterilization.

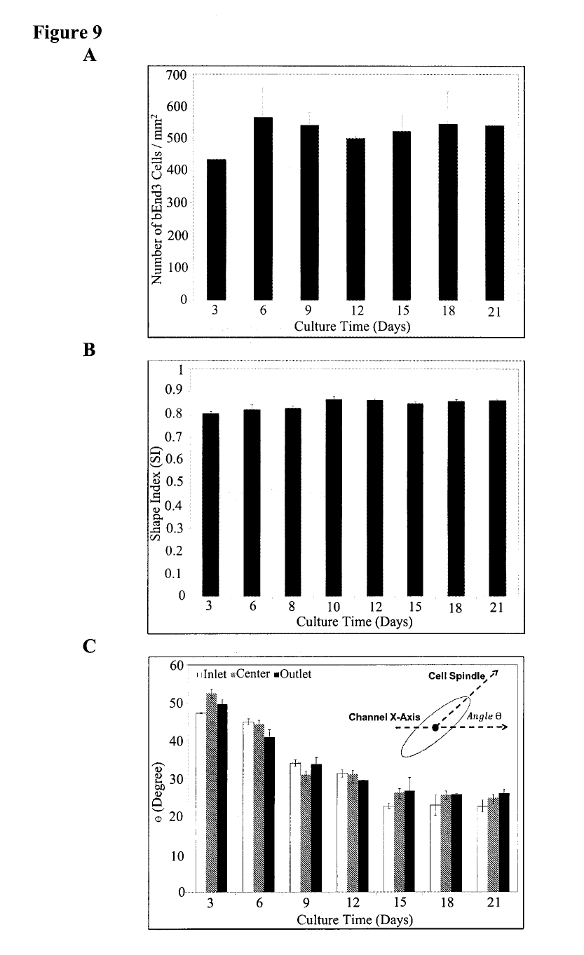

Since the microfluidic channel was designed, for example, for cell culture and validation of cell monolayer integrity (FIG. 8), cell culture protocols and parameters such as the seeding density and frequency of medium change were established. In order to determine the optimal seeding density for mouse brain endothelial cell line (b.End3), cell suspensions were made with different cell densities using flow cytometry, and each channel was seeded with a different cell suspension. Among all five seeding densities, at 24,000 cells/channel, 18,000 cells/channel, 12,000 cells/channel, 8,000 cells/channel, and 6,000 cells/channel, it was determined that 8,000 cells/channel was the optimum seeding density. Generally, a seeding density higher than 8,000 cells/channel resulted in rapid consumption of nutrients in the medium and accumulation of secreted chemicals leading to an observable pH shift that resulted in cell death; 6,000 cells/channel; on the other hand, was not sufficient for the cells to reach confluence in the channels. It was also determined that in order to optimize cell growth within the channels, medium change every 12 hours is preferred in order to prevent evaporation due to the small volume of the channels and the rapid pH shift that could potentially occur upon fluid loss.

Live/Dead Cytotoxicity Assay, Cell count, Angle Alignment, and Shape Index (SI) Measurements

The design incorporated two layered microfluidic channels that sandwich a polyester membrane filter along the complete length of the channel, which was used to culture b.End3 cells. Using live/dead cytotoxicity assay, the cells were stained and it was confirmed that b.End3 cells were successfully cultured in the layered channels. It was further determined that the confluent monolayer was maintained at least 24 days within the channels. This prolonged viability is advantageous in the long run compared to standard transwells where endothelial cells are typically used within two weeks after seeding. During the initial staining process, it was determined that some channels, due to the membrane not being able to completely cover the overlapping region of the top and bottom layers of the channels, allowed “cross-talk” between the two layers. Therefore, an additional quality control step was added using medium to test the integrity of the channels before cell seeding, any channels that were found to allow “cross-talk” were not used during the experiments.

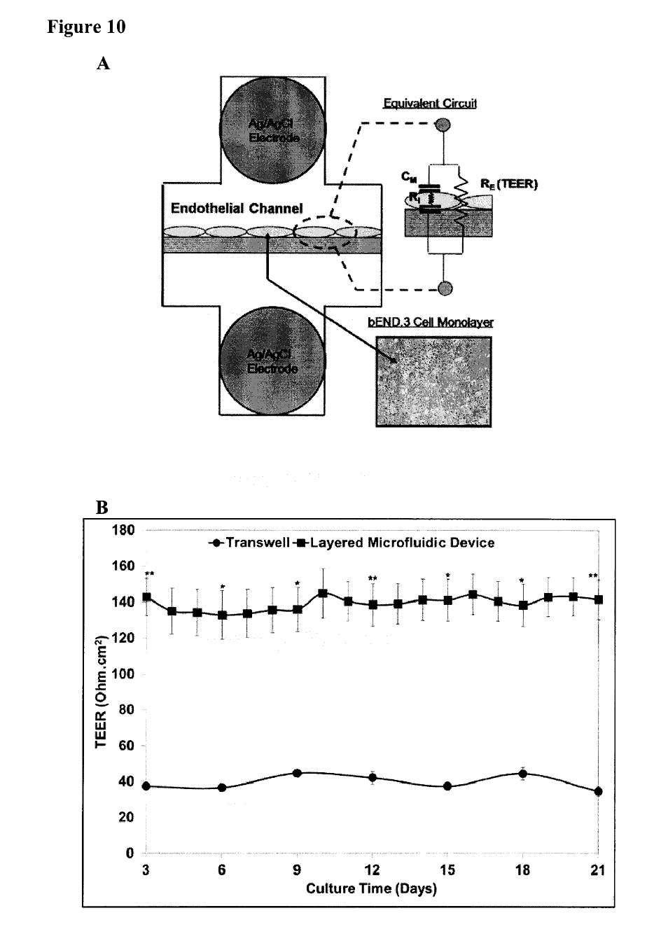

After staining the endothelial cells, fluorescent imaging was utilized to take 3 images along the length of the channels at the inlet, center, and outlet as a function of days in culture at 4× and 10× magnification up to 15 days (FIG. 8). Using the 10× images, the number of cells in each image was counted and all 3 images from day 5 to day 15 were averaged (FIG. 9). The data shows that from day 5 to day 10, the cell number was steadily increasing; however, at day 11 and day 12, a drop in cell count was observed, but the number recovered from day 13 onward. Despite the drop in cell count at day 11 and day 12, fluorescent images show that the monolayer was still in-tact and confluent. The present invention is not limited to a particular mechanism. Indeed, an understanding of the mechanism is not necessary to understand the present invention. Nonetheless, it is contemplated that the observed decrease is due to cells being over confluent inside the channels and undergoing apoptosis in order to make space available for other cells to grow. Regardless of the cell count, however, as long as the confluence of the monolayer is not affected, monolayers are suitable for use.

Using 4× images, angle alignment analysis was performed. Briefly, 300 cells were randomly selected from each image and their angle along the x-axis was measured by using the ruler tool in Adobe Photoshop 5.0. For each cell, the nucleus was selected as the center for the axis and a line was dragged from the center point to the end of the endothelial spindle. The ruler tool automatically measured the angle of this line against the x-axis and the numbers were recorded to a single decimal point. Once all images had been measured, the entire data entry was processed by plotting the average of each image as a function of time (FIG. 9). A steady decrease in the alignment angle of the cells was observed. The present invention is not limited to a particular mechanism. Indeed, an understanding of the mechanism is not necessary to understand the present invention. Nonetheless, it is contemplated that this is due to the rectangular shape of the top layer of the channels that forced the cells to align themselves steadily along the length of the channel (Velve-Casquillas et al., Nano Today 5, 28-47 (2010); Green, J. V. et al. Lab on a Chip 9, 677-685 (2009)). This observed morphological change indicates that continuous culture within layered PDMS channels results in changes in cellular structure and the associated tight junctions.

Using the same 4× images, another parameter that defines morphological response of endothelial cells, the Shape Index (SI), was measured. SI is a dimensionless measure of the spread or roundness of a cell calculated by the perimeter and area of the cell and is defined as the following:

$$SI = \frac{4 π A}{{P}^{2}} ,$$

where A is the area of the cell and P is the perimeter of the cell (Song, J. W. et al. Analytical Chemistry 77, 3993-3999 (2005)). SI can range from 0 to 1 where 0 is defined as a straight line according to the formula and 1 is defined as a circular object (Song et al., supra). To measure the SI, 100 cells were randomly selected from each image; their perimeter and area were measured by using the selection tool in ImageJ 1.43. The selection tool allows one to trace the cell perimeter one at a time; it measures and records both the perimeter and area along the trace. The same data processing procedure was used as the angle alignment measurements. No changes were observed in the SI of the endothelial cells cultured in layered microfluidic channels as a function time (FIG. 9).

### Comparative Evaluation of b.End3 Barrier Properties in Transwells and Layered Microfluidic Channels

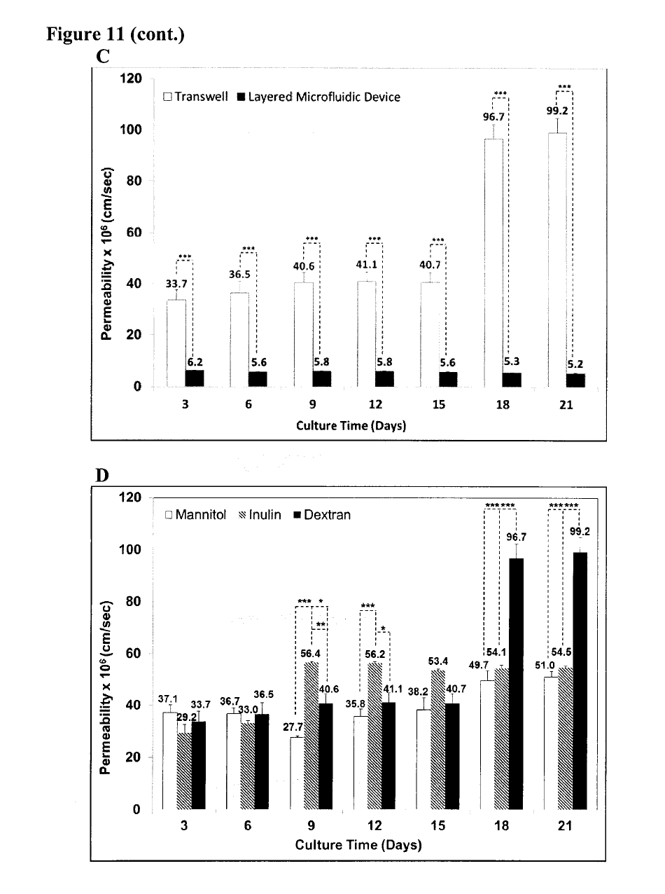

The permeability of [¹⁴C]-mannitol as a function of days across b.End3 cells cultured in transwells and layered microfluidic channels was measured. Due to mannitol's hydrophilic nature, it does not partition into the lipid bilayers of biological membranes and can only permeate across the water-filled pores in the tight junction complexes connecting adjacent epithelial and endothelial cells (Deli et al, Cellular and Molecular Neurobiology 25, 59-127 (2004)). Consequently, mannitol has been routinely used to assess the paracellular permeability of epithelial and endothelial monolayers. The results clearly show that [¹⁴C]-mannitol exhibited 15-fold lower permeability in the layered microfluidic channels compared to the transwells (FIG. 11). This indicates that the tight junctions controlling the permeability of mannitol across the paracellular route between adjacent endothelial cells were developed as early as 2 days in culture in layered microfluidic channels and they were more “restrictive” compared to the junctions developed in the transwells despite the identical seeding densities. This clearly indicates that device described herein induced the formation of a more physiological barrier compared to conventional transwell model.

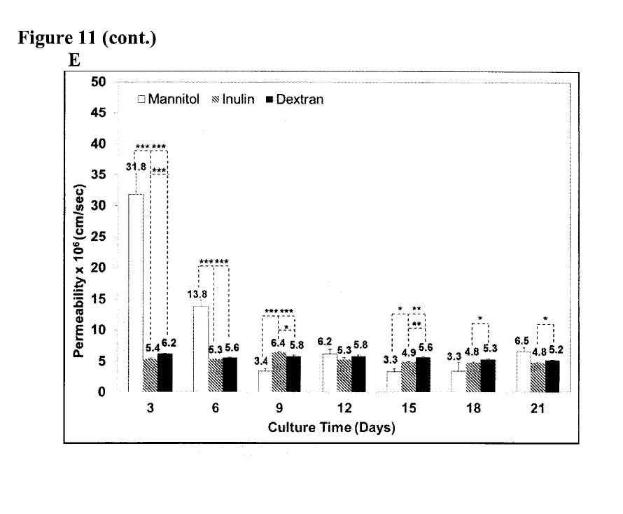

The functional expression of P-glycoprotein (P-gp) pumps that typically limit the diffusion of a wide range of hydrophobic drug molecules (e.g. anticancer drugs) across the BBB was also investigated. The apical-to-basolateral (AB) and basolateral-to-apical (BA) permeability of [³H]-dexamethasone, which is a glucocorticoid steroid hormone with a potent anti-inflammatory activity and a strong substrate of P-gp (Ueda, K. et al. The Journal of Biological Chemistry 267, 24248-24252 (1992)), across the model was investigated (FIG. 12). BA permeability is typically higher than AB permeability when P-gp is expressed in vivo. The results showed that P-gp pump started to display its activity after 12 days in culture with an efflux ratio (Permeability_{BA}/Permeability_{AB}) of 1.3. This ratio was also evident at day 15, 18, and 21 days in culture. While the existence of P-gp in the BBB is confirmed in vivo (Rubin and Staddon, Annual Review of Neuroscience 22, 11-28 (1999)), many in vitro models do not evaluate the presence of P-gp but instead only focus on the tight junctions and paracellular transport properties.

Comparative Evaluation of b.End3 mRNA Expressions in Transwells and Layered Microfluidic Channels

Quantitative real-time PCR, qPCR for short, is a technique used to amplify and quantify targeted mRNA molecules simultaneously. By using GAPDH as a reference gene, it enables one to detect and quantify several key mRNA molecules including those that translate into occludin, claudin-1, claudin-3, claudin-5, and actin, and compare their relative quantities between b.End3 cells grown in transwells and the layered microfluidic channels at day 7 in culture.

To isolate RNA from b.End3 cells, RLT buffer is prepared by using 350 μl/sample of RLT and mixed with 10 μl of β-mercaptoethanol per 1 ml of RLT needed. Medium is removed and cells are rinsed with PBS buffer solution. After PBS is aspirated, 350 μl of RLT solution with (1-mercaptoethanol is added to each well. The RLT solution is rinsed through the cell monolayer 2-3 times, and then the lysate is transferred into a pre-labeled QIA shredder. The lysate is centrifuged through the QIA shredders at maximum speed (˜13,000 rpm) for 2 minutes. 350 μl of 70% ethanol is added to the flow-through lysate and well mixed. The entire mixture (˜700 μr/sample) is then transferred to a labeled RNeasy mini column. The columns are centrifuged at 10,000 rpm for 15 seconds to allow RNA and DNA to bind to the column, and the flow-through is discarded. The columns are rinsed by adding 350 μl RW1 buffer and centrifuged at 10,000 rpm for 15 seconds; the flow-through is discarded. 80 μl of DNase per column is added and incubated for 15 minutes at room temperature. 350 μl of RW1 buffer is then added again and the columns are centrifuged at 10,000 rpm for 15 seconds; flow-through is discarded. 500 μl of RPE buffer is added to each column and centrifuged at 10,000 rpm for 15 seconds; flow-through is discarded. 500 μl of RPE buffer is added again; this time all columns are centrifuged for 2 minutes. All columns are then transferred to new collection tubes and centrifuged at 13,000 rpm for 1 minute to remove all left-over ethanol. All columns are transferred again to pre-labeled RNase-free collection tubes, 50 μl of RNase-free water is added to each column and centrifuged at 10,000 for 1 minute. 2 μl of solution is taken from each column and its concentration is measured, the unit of concentration is given in ng/μl.

To initiate reverse transcription, a master mix containing 2 μl of 10×RT buffer, 2 μl of dNTP mixture, 2 μl of hexamers, 1 μl of RNase inhibitor, and 1 μl of ORT is prepared for each sample. The 8 μl master mix is transferred into pre-labeled PCR tubes and stored on ice. Solution containing 250 ng of RNA (calculated using measured concentration) and water are added to the PCR tubes to make a final reaction volume of 20 μl. PCR tubes are vortexed and spun down; they are then incubated in the water bath at 37° C. for 1 hour. This reverse transcription reaction makes cDNA copies of the target RNA molecule.

cDNA samples are diluted by adding 6 μl of cDNA to 50.26 μl of de-ionized water. 10 μl of 2× TaqMan Master Mix, 1 μl of 20× primers, and 9 μl of diluted cDNA are added to a single well in a 96-well plate as one reaction, 3 reactions are done for each sample. After all reactions are loaded into the wells, the plate is sealed with an adhesive cover and centrifuged for 1 minute at 600 rpm. The reactions are measured using Applied Biosystems 7500 Real-Time PCR system; the thermal cycle is set at: 2 minutes at 50° C. (once), 10 minutes at 95° C. (once), and then 40 cycles of alternatives of 15 seconds at 95° C. and 1 minute at 60° C. where fluorescence is measured.

The data is analyzed by the 2^{-ΔΔCT} method (Livak and Schmittgen, Methods 25, 402-408 (2001)). Briefly, the Real-Time PCR system measures C_{T} values for each reaction, using GAPDH as the reference and the transwell model as the baseline, ΔΔC_{T} is calculated as the following:

ΔΔC_{T}−(C_{T,Target}−C_{T,GAPDH})_{PDMSModel}−(C_{T,Target}−C_{T,GAPDH})_{Transwell}

Then the change in gene expression for each reaction is calculated using 2^{-ΔΔCT}

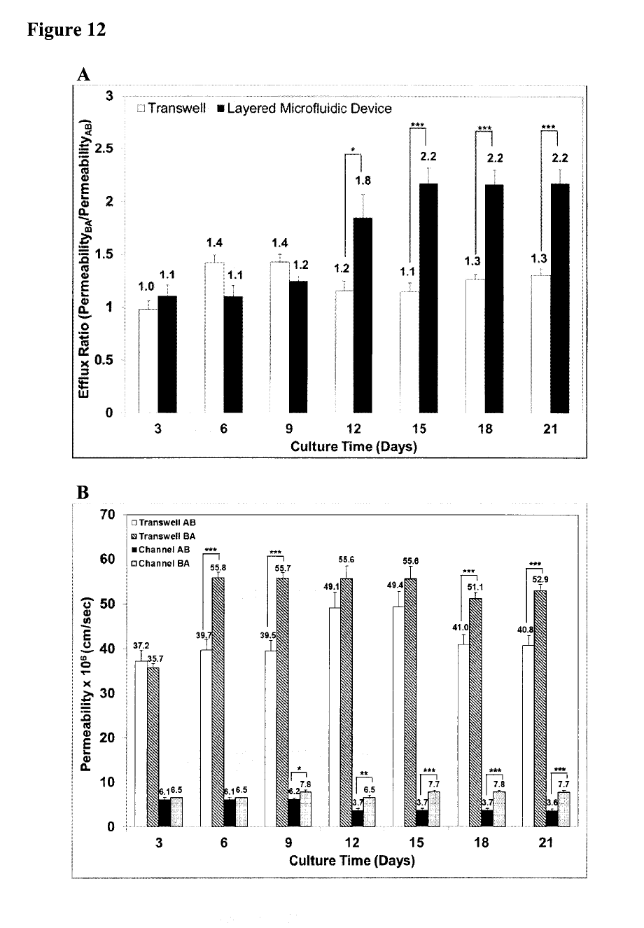

The preliminary qPCR experiment shows that there are significant changes in several tight junction proteins including occludin, claudin-1, and claudin-5 (FIG. 13). There was also an observed increase in cytoskeleton component actin, which can be contributed to the gradual angle alignment changes that occurred to the cells cultured in layered microfluidic channels. The significant increase observed in occludin and claudin-1 mRNA coincides with observed decrease of permeability of [¹⁴C]-mannitol. Occludin is generally considered the “gate keeper” of the tight junctions and guards against any paracellular molecules without any specificity. Previous work by Liebner and colleagues has shown that the down-regulation of claudin-1 can be directly linked in formation of leaky vasculature that had tight junctions with altered morphology (Liebner, S. et al. Acta Neuropathol 100, 323-331 (2000)). Therefore, the increased occludin and claudin-1 mRNA expression for the cells grown in layered microfluidic channels directly contributed to the decreased permeability of [¹⁴C]-mannitol across the cell monolayer. A decreased level of expression of claudin-5 was observed. Claudin-5 has been found to affect the size selectivity of tight junctions in vivo (Nitta, T. et al., The Journal of Cell Biology 161, 653-660 (2003)).

### Example 3

Static Microfluidic Model with b.End3 Cell Line

### Permeability of [14C]-Mannitol, [14]-Inulin, and [14C]-Dextran Across b.End3 Monolayer in Static Microfluidic Model

In order to examine the size selectivity of the tight junctions formed by the endothelial cells in the static model, three paracellular transport markers with different molecular weight (MW) are selected: [¹⁴C]-mannitol (MW=182.17), [¹⁴C]-inulin (MW=6179), and [¹⁴C]-dextran (MW=105000) (Wishart, D. S. et al. Nucleic Acids Res., D901-906 (2008)). If endothelial monolayers grown in this model possess size selectivity, it is expected that differences in permeability for these three marker molecules is observed. Specifically, mannitol is predicted to demonstrate the highest permeability across cell monolayer through paracellular transport due to it being the smallest among the selected molecules; while dextran is predicted to display the lowest permeability due to its large molecular weight and size. The permeability of inulin is likely to be between mannitol and dextran.

Permeability experiments are performed at day 3, day 12, and day 21 for all three marker molecules with five replicates for each experiment. If significant difference for each marker is found during the initial phase of the experiment as a function of days in culture, additional experiments at day 6, day 9, day 15, and day 18 are performed as necessary to pin-point when the change in permeability occurs.

All permeability experiments are performed based on established protocols (Cucullo, L. et al. Journal of Cerebral Blood Flow & Metabolism 28, 312-328 (2008)). Briefly, stock solution with 3 μl of radioactive material and 24 μl of PBS solution is prepared for each channel. The standard dilution scheme is prepared according to Table 1:

TABLE 1

| | Volume of | | |
|---|---|---|---|
| Standard | Stock Solution | Volume of PBS | Percent Dilution |
| S1 | 27 μl | 0 μl | 100% |
| S2 | 20.25 μl | 6.75 μl | 75% |
| S3 | 13.5 μl | 13.5 μl | 50% |
| S4 | 2.7 μl | 24.3 μl | 10% |
| S5 | 1.35 μl | 25.65 μl | 5% |
| S6 | 0.27 μl | 26.73 μl | 1% |
| S7 | 0.135 μl | 26.865 μl | 0.5% |
| S8 | 0.027 μl | 26.973 μl | 0.1% |

Cells grown in layered microfluidic channels under static condition are removed from the incubator and washed twice by replacing culture media with 27 μl of PBS solution. PBS solutions are aspirated and replaced by 27 μl of the stock solution at the apical (top) compartment. All channels are then placed onto the shaker at 100 rpm inside the incubator at 37° C., 95% air, and 5% CO₂ for a total of 180 minutes. At every 30 minute interval, the channels are taken out of the incubator; original PBS solutions from the basolateral compartments are collected, the basolateral compartments are then washed three times with fresh PBS, and the channels are returned to the incubator. At the end of the incubation time, the solutions in both the apical and basolateral compartments are collected and both compartments are washed with fresh PBS three times each. All the samples collected during the experiments are measured using a liquid scintillation counter, which evaluates the radioactivity of each sample in the unit of count per minute (CPM). CPMs from all samples are then converted into concentration using the standard dilution scheme.

The equation for calculating permeability of a given molecule across the monolayer can be derived from a differential equation based on Fick's Law as shown below:

Flux−PA(C_{apical}−C_{basolateral})

The equation is a modified version based on the first of Fick's laws of diffusion from a biological perspective (Cucullo et al., supra). In this equation “Flux” represents the amount of solutes entering the basolateral compartment from the apical side over time, P is the permeability coefficient, A is the surface area where diffusion occurs, C_{apical} is the concentration at the apical compartment, and C_{basolateral} is the concentration at the basolateral compartment. The equation can also be re-written as:

$$\frac{d {M}_{basolateral}}{d t} = PA ( {C}_{apical} - {C}_{basolateral} )$$

where M_{basolateral} is the amount of solutes entering the basolateral compartment and t represents time. By dividing both sides of the equation by the volume of the basolateral compartment (V_{basolateral}), the following is obtained:

$$\frac{d {C}_{basolateral}}{d t} = \frac{PA}{{V}_{basolateral}} \times ( {C}_{apical} - {C}_{basolateral} )$$

The equation above contains all parameters that can be either controlled or measured in a laboratory setting with the exception of the permeability coefficient. Since the basolateral compartment is replenished with fresh PBS at every time interval, for all practical purposes, C_{basolateral}=0; and by re-organizing the equation, the permeability equation that can be used to calculate the permeability coefficient is determined:

$$P = \frac{{V}_{basolateral} \times \frac{Δ {C}_{basolateral}}{Δ t}}{A \times {C}_{apical}}$$

The transfer of solutes from the apical to the basolateral side is not concentration dependent, and only varies as a function of time. The unit of measurement cm/s for permeability coefficient is commonly used.

### Evaluation of Tight Junction Protein Expression

Western blot are used to examine the total expression of occludin, claudin-1, claudin-5, and actin at day 3, day 12, and day 21 in culture, and compare their expressions with cells grown in the 12-well transwells.

Western blotting is performed using established protocols (Brown et al., Brain Research 1130, 17-30 (2007)). Briefly, total protein from confluent b.End3 monolayers is isolated by dissolving cells in TRIzol Reagent. Protein concentrations are then measured using bicinchoninic acid method by using bovine serum albumin as the standard. Samples of 10-20 μg are separated by electrophoresis on 4-20% gels at 125 volt for 90 minutes. Proteins are transferred to polyvinylidene fluoride membranes and are incubated with primary antibodies, such as anti-occludin, anti-claudin-1, anti-claudin-5, and anti-actin with a dilution ratio of 1:1000 in 0.5% BSA/PBS overnight at 4° C. HRP-conjugated secondary antibodies at 1:2000 dilution ratio in 0.5% BSA/PBS is applied for 1 hour at room temperature. Protein bands are then visualized by using enhanced chemiluminescent method and X-ray film. The density of protein bands is quantified using Scion Image software. All quantifiable results are normalized against GAPDH on the same film to account for variability.

### Localization of Important Tight Junction Proteins by Confocal Microscopy

The localization of three tight junction proteins (occludin, claudin-1, and claudin-5) and one cytoskeletal protein (actin) is tested at day 3, day 12, and day 21 in culture according to established protocols (Brown et al., Brain Research 1130, 17-30 (2007)). Once the specified culture time is reached, b.End3 cells are incubated in wheat germ agglutinin-tetramethyl rhodamine at a diluted ratio of 1:200 for 30-40 minutes at 4° C. to label cell membranes. Cells are fixed by using 3.7% paraformaldehyde for 10 minutes at room temperature. 0.1% Triton X is used to allow permeablization of primary antibodies at 1:100 dilution ratio in PBS with 10% donkey serum to block non-specific labeling. Cells are then incubated with Cy2-labeled donkey anti-mouse secondary antibody and anti-fade media containing DAPI counterstain for the nuclei. Fixed cells are visualized by confocal microscopy using a 63× water immersion objective. The excitation wavelengths are 488 nm and 543 nm, and emission wavelengths are 530 nm and 560 nm for Cy2 and tetramethyl rhodamine, respectively.

Due to the high magnification required for confocal microscopy, the maximum focal length (from the base of the channel to the porous membrane where the cells are grown on) of the objective is 0.7 mm; however the thickness of the basolateral compartment of the layered PDMS channels is approximately 4 mm on average. This discrepancy means that the cell monolayer cannot be observed unless the thickness is reduced by six folds. PDMS, having elastic properties, is not preferred as a rigid medium reservoir at the required thickness; therefore, a basolateral compartment using glass with the exact same configuration at a much reduced thickness is developed. This allows both regular cell culture without changing experimental parameters and visualization of the cell monolayers using confocal microscopy.

By comparing confocal images taken from both the transwells and layered PDMS channels, a baseline for the dynamic model that implements physiological shear stress into these channels is developed.

Dynamic Micro Fluidic Model with b.End3 Cell Line:
Set-Up of In Vitro Dynamic Model with Continuous Laminar Flow

Flow induced shear stress is a physiological parameter that endothelial cells experience constantly in vivo. Larger blood vessels such as the aorta and arterioles experience pulsatile flow that corresponds to heart beats that initiate blood circulation; capillaries, on the other hand, experience laminar flow due to the dampening of the pulsatile flow by gradually decreasing cross-sectional areas through branching and the assistance of smooth muscle cells and pericytes that are capable of controlling blood flow. Earlier studies have found that the endothelium responds rapidly to the mechanical conditions created by blood flow, and shear stress has the ability to modulate mechanotransducers, intermediate signaling molecules, mitogen activated protein kinases, and effector molecules such as nitric oxide (Traub and Berk, Arterioscler Thromb Vasc Biol. 18, 677-685 (1998)). Cucullo and colleagues found that shear stress plays a significant role in inducing the BBB properties in vitro, even more so than co-culturing the endothelial cells with astrocytes (Cucullo et al., supra), which has been widely believed to stimulate the development and regulation of BBB (Abbott, Journal of Anatomy 200, 629-638 (2002)).

To induce physiological shear stress, flow rate is calculated based on shear stress values found in the literature and the dimensions of the layered PDMS channels:

$$τ = \frac{6 μ Q}{{a}^{2} b}$$

where ^{τ} is the shear stress, ^{μ} is the viscosity of fluid, Q is the flow rate, a is the height of the channel, and b is the width of the channel (Song et al. Analytical Chemistry 77, 3993-3999 (2005)). By re-organizing the above equation, it becomes:

$$Q = \frac{τ {a}^{2} b}{6 μ}$$

Physiological shear stress of brain capillaries ranges from 4-20 dyne/cm² (Colgan, O. C. et al. Am J Physiol Heart Circ Physiol 292, 3190-3197 (2007). Shear stress at 4 dyne/cm² is induced. This value, while capable of providing adequate physiological shear stress, also minimizes the risk of excess mechanical force that may result in cell death. To estimate the flow rate, fluid viscosity is assumed to be similar to that of water, at 0.798 mPa s (Hawrylak et al., Journal of Solution Chemistry 27, 827-841), channel height is 0.2 mm, and channel width is 2 mm. The calculation indicates that a flow rate of 6.7 μl/s is useful to induce a physiological shear stress at 4 dyne/cm².

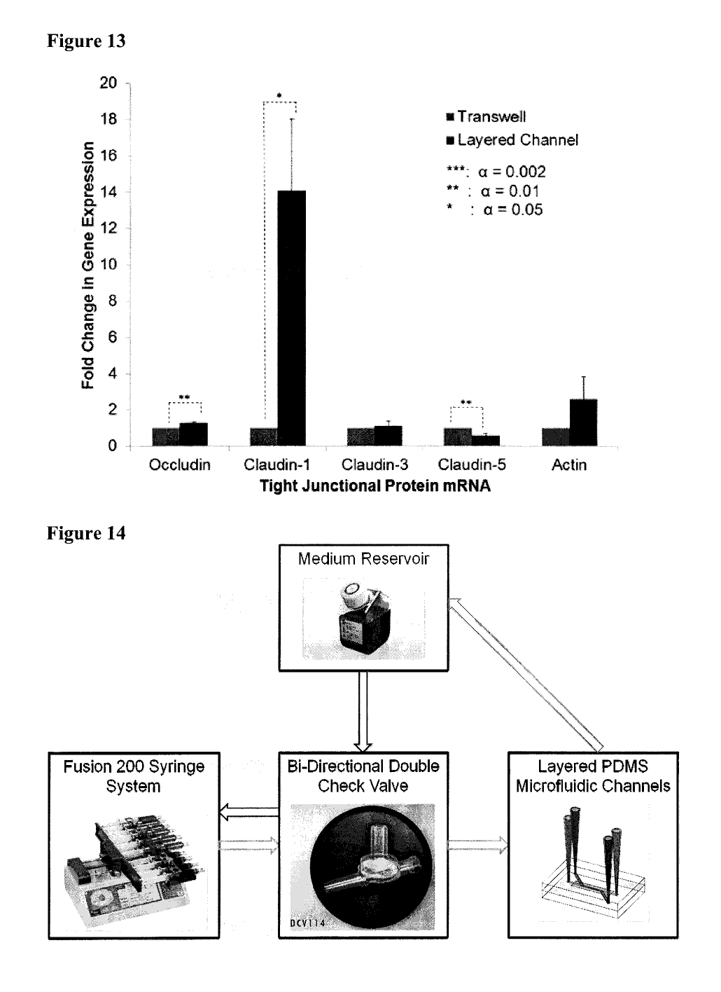

To set-up constant flow within the layered PDMS channels that is capable of inducing the flow rate for up to 21 days in culture, a recycling mechanism is implemented so that flow-induction can be self-maintained without constant supervision. A Fusion 200 Syringe Pump system that has the capability to dispense liquid in the syringe at the rates range from 0.001 μl/hour to 45 ml/min is used; 6.70 μl/s is well within the range of the dispensing power of the system. The system is connected to a bi-directional double check valve that is designed to control the directional flow of fluid. The medium reservoir is connected to the valve's supply port using connective tubing, the aspiration port is connected to the syringe system, and the exit port is connected with layered microfluidic channels. When the syringe system dispenses fluid, the valve controls flow so that it would only go through to the exit port into the layered PDMS channels; and when the syringe system withdraws fluid, the valve controls it to only withdraw from the medium reservoir thus achieving automated continuous flow (FIG. 14). The dispensing and withdrawing sequences are programmed into the Fusion 200 Syringe Pump system so that they repeat after one another. The dispensing sequence activates when the syringe is full and dispenses cell culture medium for approximately half an hour; when the syringe becomes empty, the system begins the withdrawing sequence to refill the syringe, this is programmed to take less than 5 seconds to ensure minimal interruption to the flow. Cell culture medium in the reservoir is changed every other day to ensure adequate nutrients for cell survival and growth.

### Evaluation of Barrier Properties of the Dynamic Microfluidic Model

All experiments performed on the static microfluidic model are also performed on the dynamic microfluidic model. Specifically, the morphologies of the b.End3 endothelial cells are investigated by staining the cells using live/dead cytotoxicity assay and measuring cell counts, angle alignment, and shape index. The paracellular transport properties and size selectivity of the monolayer are investigated by using permeability of [¹⁴C]-mannitol, [¹⁴C]-inulin, and [¹⁴C]-dextran at day 3, day 12, and day 21. TEER is measured by using layered microfluidic channels with embedded electrodes every 24 hours to monitor potential changes in ionic flow of the tight junctions. The permeability and TEER measurements are compared to the ones measured in the static model; if significant changes are found, western blot and protein localization experiments of occludin, claudin-1, claudin-5, and actin are performed and compared to the static model for similarities and differences.

The present invention is not limited to a particular mechanism. Indeed, an understanding of the mechanism is not necessary to understand the present invention. Nonetheless, it is contemplated that since pervious work has indicated that flow alone can alter the permeability of endothelial cell monolayers (Cucullo et al., supra), cell monolayers in the dynamic model have better angle alignment and shape index, lowered permeability values on all marker molecules, more protein expressions, and more physiological localization of these proteins compared to the static model.

Dynamic Microfluidic Model with HCMEC Cell Line:

### Evaluation of Barrier Properties of the Dynamic Microfluidic Model

Human brain vascular endothelial cell line (HCMEC/D3) has been found to induce BBB-like properties without the need for astrocyte co-culture (Cucullo et al., supra). The dynamic model is assayed for its capability to induce BBB-like properties in this cell line as well as b.End3 and compare the barrier properties between the two.

Permeability of [¹⁴C]-mannitol is used to evaluate the paracellular transport properties of the monolayer, and localization studies of occludin, claudin-1, claudin-5, and actin by confocal microscopy is used to study the co-localization of important proteins to the formation of tight junctions and endothelial morphology.

### Model Comparison and Evaluations of P-gp and Transporters:

### Data Comparison Among Transwell, Static Microfluidic, and Dynamic Microfluidic Models

The transwell, static microfluidic, and dynamic microfluidic models are compared based on cell viability, morphology, paracellular permeability, TEER measurements, protein expression, and localization of tight junction proteins, and the model that best mimics the in vivo BBB is identified. The quantitative permeability of marker molecules is an important part of the data due to its direct measurable parameters, such as time and concentration. TEER measurements also provide a quick and easy way to evaluate of the integrity of cell monolayers. The morphological measurements, such as cell count, angle alignment, and shape index provide evidence of cellular morphological changes of the cells induced by the design of the models. The presence and localization of important tight junction proteins by ways of Western blot and confocal microscopy are useful to explain the permeability and morphological changes that are observed in the models.

### Evaluation of P-Glycoprotein (P-gp) Expressions

Even though the integrity of cell monolayers via paracellular transport is an important indicator of BBB transport properties, it also possesses other indicators such as important transporters that also contribute to the restricted transport properties of BBB in vivo. One important transporters is an efflux pump called P-glycoprotein, or P-gp (Rubin and Staddon, Annual Review of Neuroscience 22, 11-28 (1999)). P-gp is a membrane-bound protein that actively exports lipid-soluble molecules that have diffused into the cells through the lipid-bilayer back into the blood stream; it has hundreds of potential substrates, many are important therapeutic agents. With its presence at the BBB in vivo, many hydrophobic molecules are unable to cross the barrier into the brain; thus a physiologically-relevant in vitro model of the BBB preferably possesses functional P-gp.

Western blot is used to confirm the presence of P-gp. The functionality of P-gp can be evaluated by using a bi-directional permeability experiment. Both the apical-to-basolateral permeability (Permeability_{AB}) and the basolateral-to-apical permeability (Permeability_{BA}) are evaluated. Radioactively labeled [3H]-dexamethasone, a strong substrate of P-gp, is used for this permeability experiment. Once the data has been processed, the efflux ratio is calculated by dividing the Permeability_{BA} by Permeability_{AB}. An efflux ratio larger than 1 indicates that functional P-gp is present in the monolayer.

### Evaluation of Other Transporters: GLUT-1 and LAT-1

To evaluate transporter mediated transport, GLUT-1, which is a glucose transporter at the BBB, and LAT-1, which transports neutral amino acids, is evaluated. The uptake studies are performed according to established protocols (Omidi, Y. et al. Brain Research 990, 95-112 (2003)). Briefly, the concentration-dependent experiments used to determine Kₘ and Vₘₐₓ of the uptake model are conducted at 5 mM, 75 mM, 150 mM, and 200 mM of D-glucose and 1 μM, 75 μM, 150 μM, and 200 μM of L-leucine. Each sample is loaded into each channel for 10 minutes. At the end of the experiment, cells are disaggregated by flushing the channels with PBS and washing the cells with 10 mM EDTA for 20 minutes. Cells are then centrifuged at 10,000×g for 10 minutes to remove any extracellular D-glucose or L-leucine. The cell pellet is disrupted with 200 μl of 0.5 M NaOH. The radioactivity of the samples is then measured by scintillation counting. Kₘ and Vₘₐₓ are calculated using the Eadie-Hofstee equation with linearized data:

$$\frac{d S}{d t} = {V}_{max} - \frac{[ \frac{d S}{d τ} ] \times {K}_{m}}{S}$$

where S is the substrate concentration. Alternatively, the Michaelis-Menten equation for non-linearized data is used:

$$v = \frac{{V}_{max} \times [ S ]}{{K}_{m} + [ S ]}$$

### Example 4

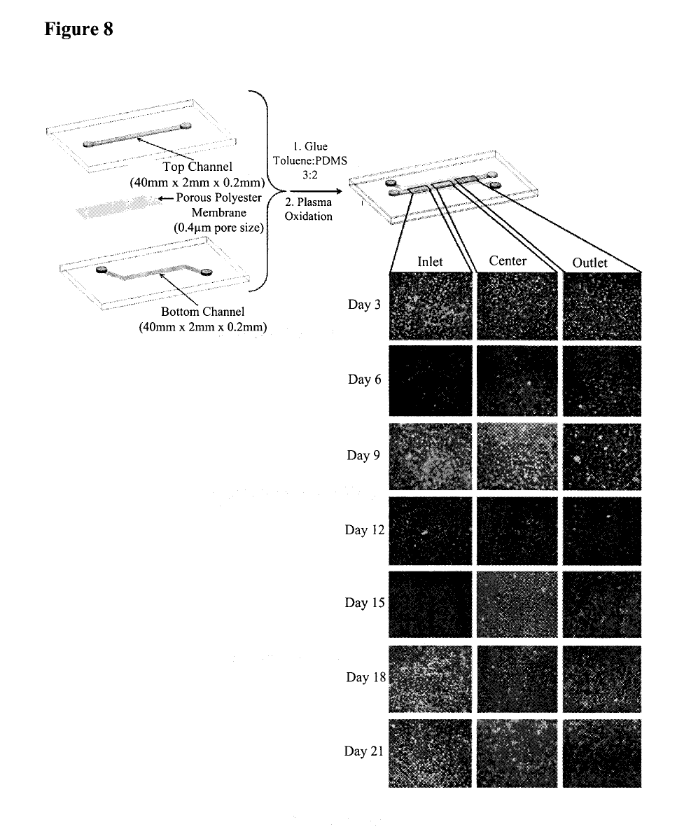

Co-culture Microfluidic Model with b.End3 Cell Line. The devices described herein find use in co-culture models. Supporting neural cells such as astrocytes, pericytes, neurons, or microglial cells are cultured on the opposite side of b.End.3 cells cultured onto semi-porous membranes or in the lower compartment of the microfluidic devices (FIG. 7). This configuration allows the in vitro modeling of interactions of various cell types with b.End.3 cells to develop a physiologically-relevant in vitro model of the BBB. To seed the supporting neural cells into the microfluidic device, these cells are loaded into the bottom chamber of the microfluidic device followed by flipping the entire device up-side-down for 12 hours to allow cell adhesion. During this time, the inlets and outlets of the device are sealed using parafilm to allow cell attachment. Once the seeding on the bottom side of the membrane is complete, the channel is flipped back to allow seeding of b.End.3 endothelial cells onto the semi-porous membrane in the top channel (FIG. 7).

Another configuration is to culture a second cell type in the bottom chamber (FIG. 7B); while this configuration does not allow direct interactions between different monolayers to occur, it enables the study of relevant soluble cell-to-cell signaling factors secreted by the monolayers in a more physiologically relevant fashion. To seed the second cell type, simply flow cells into the bottom chamber of the channel with sufficient densities for cells to attach and proliferate.

These two co-culture configurations can be combined to create a triple culture model where the primary cell type (e.g., b.End3 cells) is cultured in the top chamber on the porous membrane, the second cell type is cultured on the opposite side of the membrane (e.g., pericytes), and a third cell type is cultured in the bottom chamber (e.g., astrocytes or neurons) (FIG. 7C). This configuration allows the studies of complex cellular systems such as the blood brain barrier where endothelial cells are found to frequently interact with many other cell types.

These co-culture systems are developed at identical cell seeding densities, cultured in similar media, incubated at 37° C., and gassed with 5% CO₂/95% air to maintain the medium pH at 7.4 while changing the culture medium every 12 hours. The advantage of such co-culture configurations is that the increased complexity of the culture system does not hinder the cellular assays and transport studies described herein (e.g. permeability, qPCR), while they still allow the physiological mimicry of complex in vivo cellular systems at the same time. This device provides a very convenient platform for studying multi-cell-to-cell interactions in vitro that were very difficult to study before.

### Example 5

### Materials

Poly(dimethylsiloxane) (Sylgard 184) was purchased from Dow Corning (Midland, Mich.). SU-8 50 was purchased from MicroChem (Newton, Mass.). Toluene and sterile-filtered fibronectin solution were purchased from Sigma-Aldrich (St. Louis, Mo.). [¹⁴C]-D-Mannitol (100 μCi/ml), [¹⁴C]-Inulin Methoxy (5 mCi/g), and [14C]-Methyl Dextran (2.5 mCi/g) were purchased from Moravek Biochemicals and Radiochemicals (Brea, Calif.). [3H]-Dexamethasone (1 mCi/ml) were purchased from American Radiolabeled Chemicals, Inc. (St. Louis, Mo.). A vial of mouse brain endothelial cells (b.End3) was purchased from ATCC (Manassas, Va.). T-25 and T-75 cell culture flasks and 12-well transwells with polyester membranes (500 nm pore size) were purchased from Fisher Scientific (Waltham, Mass.). Dulbecco's modified eagle medium (11005-065), fetal bovine serum (10437-028), PBS (10010-023), 0.05% trypsin (25300-054), antibiotic-antimycotic (15240-062), and live/dead cytotoxicity kit for mammalian cells were purchased from Invitrogen (Carlsbad, Calif.).

### Design and Fabrication of Layered Microfluidic Devices

Layered microfluidic channels were fabricated using soft lithography. Briefly, PDMS prepolymer was mixed with curing agent at a weight ratio of 10 (prepolymer):1 (curing agent) and was cast onto two 4 inches silicon wafer containing positive relief pattern 200 μm thick, one for the top layer and one for the bottom layer. The mixture was cured at 60° C. for 2 hours, then the cured PDMS layer was peeled from the silicon wafer. Access holes were punched with a 16 gauge blunt syringe (1.65 mm outer diameter) forming the inlet and outlet holes for each channel. In order to glue the top and the bottom layers together, PDMS mortar layers were created using PDMS and toluene mixed at 3 (PDMS):2 (toluene) weight ratio. The mixture was well mixed using a vortex mixer and it was allowed to sit for 5 minutes to remove bubbles. The toluene diluted PDMS prepolymer was then spin-coated on a clean glass cover slide for 1 minute to generate a thin mortar layer. The PDMS substrates were placed onto the glass slide spin-coated with adhesive PDMS mortar and allowed to stay in contact for 30 seconds. Polyester membranes with 200 nm pore size were used to cover the overlapping region of the top and bottom layers and both layers were aligned and glued together. The combined pieces were cured again for 1 hour until the PDMS mortar was completely hardened. 100 μl pipette tips were inserted into the inlets and outlets, acting as medium reservoir for the channel; they were then glued to the channel with PDMS. Before use, the layered PDMS channels were exposed to plasma oxygen for 5 minutes and fibronectin solution was flowed through the channels at 25 μg/ml. The solution was allowed to stay inside the channels under UV for 24 hours for coating and sterilization.

Layered microfluidic channels with embedded electrodes for measuring TEER were fabricated following established protocols. Briefly, PDMS prepolymer mixed at a weight ratio of 10 (prepolymer):1(curing agent) is poured onto the mold and is allowed to cure overnight. The layers are then removed and reservoir holes were punched to allow access to both the upper and lower channels. Ag/AgCl recording electrodes are embedded into a 500 μm×500 μm side channel. A semi-porous polyester membrane with 400 nm pore size is sandwiched between the upper and lower channels to completely cover the overlapping region. The entire channel is then sealed by using a PDMS mortar solution with weight ratio of 3 (toluene):2 (PDMS). After sealing the layered channels, PDMS prepolymer at a weight of 3 (prepolymer):1 (curing agent) is injected into the side channel. The PDMS is allowed to cure for 24 hours at room temperature.

### Culture of Mouse Brain Endothelial Cells (b.End3)

Cells in cryo-vial were thawed by incubating in a 37° C. water bath for 3 minutes. The cells were transferred to a 15 mL centrifuge tube with 3 mL of additional medium and centrifuged at 1000 rpm for 3 minutes. The supernatant was aspirated and the cell pellet was re-suspended in 10 mL of medium. The cell suspension was transferred into a T75 flask and incubated in a humidified 5% CO₂ incubator at 37° C. Cell culture medium was completely changed every 48 hours.

When cell cultures were confluent, medium was aspirated from the flasks containing the confluent monolayer, the adherent cells were washed with 10 mL of sterile PBS twice. Cells were then incubated with 5 mL of 0.05% Trypsin-EDTA solution for 3 minutes at 37° C. When cells have detached, the cell suspension was centrifuged at 1000 rpm for 3 minutes. Cell suspension was aspirated and replaced by 10 mL of culture medium. One T-75 flask with confluent monolayers was split into 5 new T-75 flasks.

The same method was used for seeding cells into 12-well transwells and layered microfluidic devices. The transwells and layered microfluidic devices were pre-treated with fibronectin solution at a concentration of 20 μg/mL. When the cell pellet was re-suspended with 10 mL of new culture medium, the cells were counted by using flow cytometry. The seeding densities were kept consistent at ˜270 cells/mm² for both the transwells and the microfluidic devices

### Characterization of Viability and Morphology of b.End3 Cells

b.End3 cells cultured in layered microfluidic devices were stained by using live/dead cytotoxicity kit for mammalian cells. Briefly, calcein AM, ethidium homodimer-1, and culture medium were mixed at 1 μL:1 μL:1 μL ratio. 16 μL of mixed solution was introduced to the cells and they were incubated for 20 minutes at 37° C. Fluorescent microscopy was used to visualize live and dead cells at 500 nm and 600 nm wavelength, respectively. Images of the cells at the inlet, center, and outlet of the layered microfluidic devices were taken at both 4× and 10× magnifications. Live and dead cell images were overlaid to produce a composite image where the green region indicates live cells and the red region indicates dead cells. Fluorescent images were taken at 3, 6, 9, 12, 15, 18, and 21 days in culture.

Cell counts were measured with a standard mechanical counter by using the 10× magnification images at the inlet, center, and outlet every three days from day 3 to day 21. Shape indexes were calculated by first measuring the perimeter and the area of each endothelial cell imaged in 4× magnification. The measurements were made using ImageJ 1.44p. The perimeter and area values were then used to calculate the shape index as the following:

$$SI = \frac{4 π A}{{P}^{2}}$$

The angel alignment of the endothelial cells along the length of the layered microfluidic channels were measured in 4× magnification images using Photoshop CS4. For each day in culture, 600 cells were selected randomly and measured independently.

### Assessment of Paracellular Permeability Across b.End3 Cell Monolayers

Three paracellular transport markers with different molecular weight (MW) are selected: [¹⁴C]-mannitol (MW=182.17), [14C]-inulin (MW=6179), and [14C]-dextran (MW=105000). Permeability experiments were performed at 3, 6, 9, 12, 15, 18, and 21 days in culture for all three marker molecules with five replicates for both the transwells and the layered microfluidic devices. Briefly, cells grown in both the transwells and the layered microfluidic devices under static condition were removed from the incubator and washed twice by replacing culture medium with PBS solution. PBS solutions were then aspirated and replaced by the designated solution with radioactively labeled marker molecules at the apical (top) compartment. All transwells and layered microfluidic devices were then placed onto the shaker at 100 rpm inside the incubator at 37° C., 95% air, and 5% CO₂ for a total of 60 minutes. At every 10 minute interval, the transwells and the layered microfluidic devices were taken out of the incubator; the PBS solutions from the basolateral compartments were collected and replaced by fresh PBS solutions. At the end of the 1 hour incubation time, the solutions in both the apical and basolateral compartments were collected. All the samples collected during the experiments were measured using liquid scintillation counter which evaluates the radioactivity of each sample in the unit of count per minute (CPM). CPMs from all samples are then converted into concentration using the standard dilution scheme.

The permeability of a given molecule across the monolayer was calculated by suing a derived form of a differential equation based on Fick's Law:

$$P = \frac{{V}_{basolateral} \times \text{?}}{A \times {C}_{apical}}$$

$$\text{?} \text{indicates text missing or illegible when filed}$$

Note that the transfer of solutes from the apical to the basolateral side is not concentration dependent, and only varies as a function of time. The unit of measurement cm/s for permeability coefficient is commonly used.

### Assessment of the Trans-Endothelial Electrical Resistance (TEER) of b.End3 Cell Monolayers

The electrical resistance values of confluent cell monolayers grown in the transwells were measured using the standard chopstick electrodes. TEER values, in terms of Ωcm², were determined by subtracting the resistance of blank filters with no cells from sample measurements. It is then standardized by the area of the filter in cm². Similarly, impedance spectra are taken using an Autolab potentiostat/galvanostat within layered microfluidic channels. The control impedance spectra measured without cells are subtracted from the measured impedance spectra with cells to eliminate any effect from electrolytes, the membrane, and the electrodes. The value is then normalized for the surface area of cell monolayers to yield TEER values in units of 11 cm².

### Assessment of Functional P-Glycoprotein in b.End3 Cell Monolayers

P-glycoprotein functionality was assessed by using permeability experiments similar to that for paracellular transport. All experimental conditions for evaluating the paracellular transport were used for P-glycoprotein assessment. [3H]-Dexamethasone was selected as the marker molecule since it is a strong substrate to the protein; it is capable of clearly demonstrating whether functionality is present in the b.End3 cell monolayers. In addition to the apical-to-basolateral permeability, basolateral-to-apical permeability was also evaluated where the radioactively labeled solution was put in the bottom compartment, and the PBS solutions from the top compartment were collected. Efflux ratio was calculated by using basolateral-to-apical permeability divided by the apical-to-basolateral permeability. If P-glycoprotein functionality was present, the efflux ratio should be higher than 1.

### Results

The design of the blood-brain barrier model incorporated two layered microfluidic channels that sandwich a polyester membrane filter along the complete length of the channel, which was used to culture b.End3 cells (FIG. 8). Using live/dead cytotoxicity assay, it was possible to stain these endothelial cells and confirm that b.End3 cells can be successfully cultured in such layered channels made of entirely by PDMS. It was further determined that the confluent monolayer can be maintained by at least 21 days within the channels, and it can be extended if necessary. This prolonged viability is advantageous in the long run compared to the standard transwells where endothelial cells can only be used within a narrow range of days in culture (day 7-day 12). During the initial staining process, it was determined that small amount of channels, due to the membrane not being able to completely cover the overlapping region of the top and bottom layers of the channels, allowed “cross-talk” between the two layers. This could compromise the integrity of the confluent cell monolayers and the accuracy of any quantitative assays performed using the channels such as the permeability experiments; therefore, an additional quality control step was added by using cell culture medium to test the integrity of the channels before cell seeding, any channels that were found to allow “cross-talk” were not used during any experiments.

After staining the b.End3 cell monolayers using live/dead cytotoxicity assay, fluorescent imaging was used. Three images were taken along the length of the channels near the inlet, center, and outlet as a function of days in culture at both 4× and 10× magnifications from day 3 to day 21 (FIG. 8). Using the 10× images, the number of cells in each image was counted and all 3 images were averaged every three days. The measurements show that from day 3 to day 6, the cell number has increased; however, after day 6, the cell counts were reasonably consistent throughout the 21 days that the cells were cultured (FIG. 9A). Fluorescent images also show that the monolayers are confluent and in-tact up to 21 days.

Using images taken at 4× magnifications, Shape Index (SI) which defines morphological characters of endothelial cells, was performed. SI is a dimensionless measurement of the spread or roundness of a cell calculated by the perimeter and area of the cell and is defined as the following:

$$SI = \frac{4 π A}{{P}^{2}}$$

where A is the area of the cell and P is the perimeter of the cell. SI can range from 0 to 1 where 0 is defined as a straight line according to the formula and 1 is defined as a perfect circle. As shown in FIG. 9B, SI values of the b.End3 cells are consistent from day 3 to day 21; this again confirms that the endothelial cells cultured inside the channels are maintained consistently throughout the entire culture time.

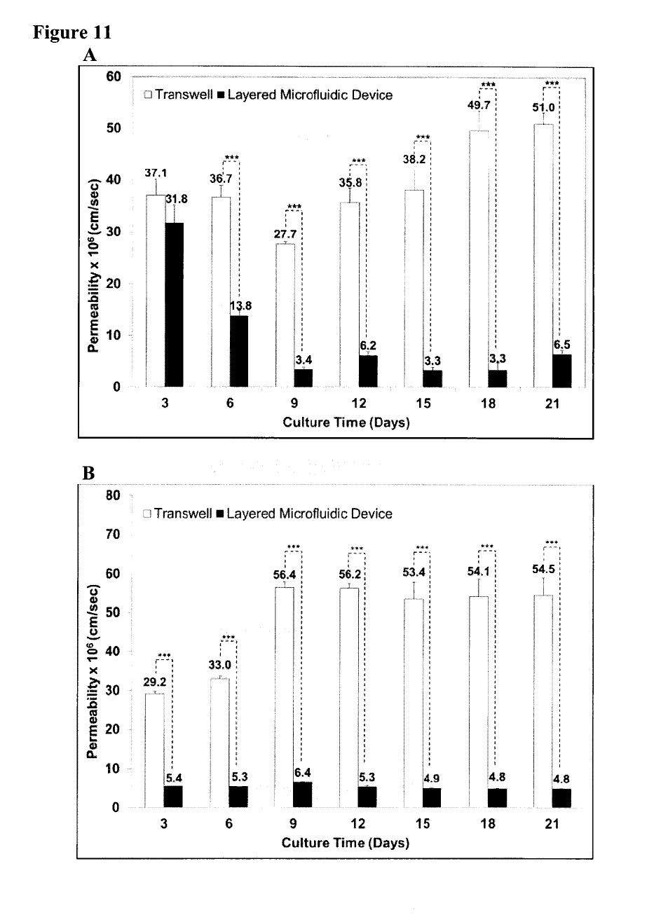

Using the same 4× images, angle alignment analysis was performed. A steady decrease in the alignment angle of the cells as a function of days in culture was observed (FIG. 9C). At day 3, the angle alignment is approximately 50° against the x-axis of the channels. This indicates that the cells cultured within the channels were randomly aligned and no uniform directionality was found. At day 21, the angle alignment of the cells becomes approximately 28°. This indicates that over the culture period of 21 days, the cells are slowly becoming aligned along the length of the channels. The present invention is not limited to a particular mechanism. Indeed, an understanding of the mechanism is not necessary to practice the present invention. Nonetheless, it is contemplated that the geometry of the channels plays a role in the observed decrease of angle alignment. It also indicates that by culturing b.End3 cells inside layered microfluidic devices, it is possible to change the morphology of the cell population, which has significant impact in the formation of cellular structures and their associated tight junctions. In addition to the characterization of cell morphology, trans-endothelial electrical resistance (TEER) across b.End3 cells cultured in both the transwells and the layered microfluidic devices was measured. The TEER values in transwells were obtained by using the standard chopstick electrodes; while Ag/AgCl electrodes were embedded into the microfluidic system to measure TEER values (FIG. 10A). The results show that the TEER values across b.End3 cells cultured in microfluidic devices were 3.5-fold higher than the resistance measured across monolayers established in conventional transwells (FIG. 10B). This indicates that the cells cultured in the microfluidic devices are better at restricting ionic flow across the monolayers than those cultured in transwells; and the higher TEER values are indicative of better formation of tight junctions. To further investigate the formation of tight junctions demonstrated by the TEER measurements, the paracellular transport of b.End3 monolayers cultured in layered microfluidic channels was compared to those established in regular transwells by measuring the permeability of [¹⁴C]-mannitol, [¹⁴C]-inulin, and [¹⁴C]-dextran (all are common paracellular permeability markers) as a function of culture time. The results show 10-fold lower [¹⁴C]-mannitol permeability across b.End3 monolayers in layered microfluidic devices compared to its permeability across monolayers established in the transwells after 9 days in culture (FIG. 11A), which indicates the formation of restrictive tight junctions between b.End3 cells cultured in the microfluidic devices compared to conventional transwells despite identical culture and experimental conditions. Inulin and dextran permeability also demonstrated a similar trend; however due to their large molecular weight, the permeability difference between the layered microfluidic devices and the standard transwells becomes significant as early as day 3 (FIGS. 11B & 11C). The permeability values also indicated an increase in the measurement at day 9 and day 18 for inulin and dextran, respectively; likely due to the disruption or discontinuity of the cell monolayers cultured in the transwells at which point the cell culture can no longer be functional as a BBB model. Cosistent permeability measurements were observed for both inulin and dextran in the microfluidic devices, which further indicates the capability of the devices maintaining stable confluent cell monolayers for up to 21 days.

By comparing the permeability data in only transwells for [¹⁴C]-mannitol, [¹⁴C]-insulin, and [¹⁴C]-dextran, it was found that the cells cultured in the transwells are not able to distinguish among the three different probes despite their differences in molecular weight until day 9 in which case the monolayers were already beginning to fail indicated by higher inulin and dextran permeability (FIG. 11D). On the other hand, cells cultured in layered microfluidic devices possess size selectivity at early culture time points such as day 3 and day 6 where small molecules such as mannitol exhibited higher permeability than large molecules such as inulin and dextran (FIG. 11E). However, this selectivity disappears after day 9 where all permeability values become very similar. This indicates that during early stages of the cell culture in layered microfluidic devices, the cell monolayers, specifically the tight junctions, were able to keep the permeability of inulin and dextran low, but small molecules such as mannitol can permeate across very effectively. As the monolayers develop, the tight junctions began to gain the ability to also regulate the permeability of mannitol, and this low permeability profile is kept consistently up to 21 days.

The functional expression of P-glycoprotein (P-gp) efflux pump was analyzed by measuring the apical-to-basolateral (AB) and basolateral-to-apical (BA) permeability of [³H]-dexamethasone which is a strong substrate for P-gp across b.End3 monolayers cultured in both the standard transwells and the layered microfluidic devices. The results show that the cell monolayers cultured in layered microfluidic devices exhibit higher efflux ratios (Permeability_{BA}/Permeability_{AB}) for b.End3 monolayers cultured for 15 days in layered microfluidic channels compared to transwells (FIGS. 12A and 12B), which indicates a statistically higher expression of functional P-gp pump by b.End3 when cultured in the microfluidic devices. This is a significant improvement over existing in vitro BBB Models, which generally fail to express different transporters including the P-gp pump. These results collectively indicate that the model provides a single-culture BBB model that is more restrictive than existing in vitro models.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in electrical engineering, optics, physics, and molecular biology or related fields are intended to be within the scope of the following claims.

We claim:

1. A system, comprising:
a) a device comprising: i) a plurality of microfluidic channels; ii) a membrane located internal to said microfluidic channels; and iii) a plurality of electrodes embedded in said microfluidic channels; and
b) a plurality of first cells adhered to said membrane or said microfluidic channels.

2. The system of claim 1, wherein said first cells are endothelial cells.

3. The system of claim 2, wherein said endothelial cells are brain endothelial cells.

4. The system of claim 3, wherein said brain endothelial cells are selected from the group consisting of b.End3 cells and human brain vascular endothelial cells (HCMEC/D3).

5. The system of claim 1, wherein said cells are growing in a monolayer.

6. The system of claim 1, wherein said system further comprises a circulatory component configured to circulate fluid through said microfluidic channels.

7. The system of claim 6, wherein said circulatory component is a pump.

8. The system of claim 7, wherein said pump further comprises valve that control the direction and/or speed of the flow of fluid.

9. The system of claim 6, wherein said circulatory component further comprises a reservoir for storing fluids.

10. The system of claim 6, wherein said fluid is a buffer.

11. The system of claim 1, wherein said electrodes are Ag/AgCl electrodes.

12. The system of claim 1, wherein said system further comprises a test compound.

13. The system of claim 12, wherein said test compound is a drug that functions in the central nervous system.

14. The system of claim 1, wherein said membrane is a semi-porous membrane.

15. The system of claim 1, further comprising a plurality of second cells, wherein said second cells are a different cell type than said first cells.

16. The system of claim 15, wherein said second cells are selected from the group consisting of astrocytes, glial cells, microglia and neurons.

17. The system of claim 15, wherein said second cells are cultured in a different portion of said device that said first cells.

18. A method, comprising:
a) contacting a device comprising a plurality of microfluidic channels, a membrane located internal to said microfluidic channels; and a plurality of electrodes embedded in said microfluidic channels with a plurality of cells, wherein said cells adhere to said membrane; and
b) culturing said cells, wherein said cells form a monolayer on said membrane.

19. The method of claim 18, further comprising the step of measuring TEER across said membrane.

20. The method of claim 18, further comprising the step of contacting said membrane with a test compound.

21. The method of claim 18, wherein said test compound is a drug that functions in the central nervous system.

22. The method of claim 21, further comprising measuring the transport of said test compound across said membrane.

23. The method of claim 18 further comprising culturing a plurality of second cells, wherein said second cells are a different cell type than said first cells.

24. The method of claim 23, wherein said second cells are selected from the group consisting of astrocytes, glial cells, microglia and neurons.

25. The method of claim 23, wherein said second cells are cultured in a different portion of said device that said first cells.
